SEQUENCE LISTING (36 sequences)

SEQ ID NO: 1 - protein, 389 aa, Artificial Sequence
  Synthetic
  misc_feature (312)..(342): Xaa can be any naturally occurring amino acid
MTMTRLKISKTLLAVMLTSAVATGSAYAENNAQTTNESAGQKVDSSMNKVGNFMDDSAITAKVKAALVDHDNIKSTDISVKTDQKVVTLSGFVESQAQAEEAVKVAKGVEGVTSVSDKLHVRDAKEGSVKGYAGDTATTSEIKAKLLADDIVPSRHVKVETTDGVVQLSGTVDSQAQSDRAESIAKAVDGVKSVKNDLKTKMGHHHHHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIXXXXXXXXXXXXXXXXXXXXXXXXXXXXXXXDGGSSGGSGGDGSSGDGGSDGDSDGSDGDGDSDGDDGGDDEDDGSDD

SEQ ID NO: 2 - protein, 31 aa, Artificial Sequence
  Synthetic
GGGGSSGGSGGSGSSGDGGSSGGSGGSGSSG

SEQ ID NO: 3 - protein, 31 aa, Artificial Sequence
  Synthetic
YYYGSSGGSGGSGSSGDGGSSGGSGGSGSSG

SEQ ID NO: 4 - protein, 31 aa, Artificial Sequence
  Synthetic
GGYYYSGGSGGSGSSGDGGSSGGSGGSGSSG

SEQ ID NO: 5 - protein, 31 aa, Artificial Sequence
  Synthetic
GGGGYYYGSGGSGSSGDGGSSGGSGGSGSSG

SEQ ID NO: 6 - protein, 31 aa, Artificial Sequence
  Synthetic
GGGGSSYYYGGSGSSGDGGSSGGSGGSGSSG

SEQ ID NO: 7 - protein, 31 aa, Artificial Sequence
  Synthetic
GGGGSSGGYYYSGSSGDGGSSGGSGGSGSSG

SEQ ID NO: 8 - protein, 31 aa, Artificial Sequence
  Synthetic
GGGGSSGGSGYYYSSGDGGSSGGSGGSGSSG

SEQ ID NO: 9 - protein, 31 aa, Artificial Sequence
  Synthetic
GGGGSSGGSGGSYYYGDGGSSGGSGGSGSSG

SEQ ID NO: 10 - protein, 31 aa, Artificial Sequence
  Synthetic
GGGGSSGGSGGSGSYYDGGSSGGSGGSGSSG

SEQ ID NO: 11 - protein, 31 aa, Artificial Sequence
  Synthetic
GGGGSSGGSGGSGSSGDYYSSGGSGGSGSSG

SEQ ID NO: 12 - protein, 31 aa, Artificial Sequence
  Synthetic
GGGGSSGGSGGSGSSGDGYYYGGSGGSGSSG

SEQ ID NO: 13 - protein, 31 aa, Artificial Sequence
  Synthetic
GGGGSSGGSGGSGSSGDGGSYYYSGGSGSSG

SEQ ID NO: 14 - protein, 31 aa, Artificial Sequence
  Synthetic
GGGGSSGGSGGSGSSGDGGSSGYYYGSGSSG

SEQ ID NO: 15 - protein, 31 aa, Artificial Sequence
  Synthetic
GGGGSSGGSGGSGSSGDGGSSGGSYYYGSSG

SEQ ID NO: 16 - protein, 31 aa, Artificial Sequence
  Synthetic
GGGGSSGGSGGSGSSGDGGSSGGSGGYYYSG

SEQ ID NO: 17 - protein, 31 aa, Artificial Sequence
  Synthetic
GGGGSSGGSGGSGSSGDGGSSGGSGGSGYYY

SEQ ID NO: 18 - protein, 31 aa, Artificial Sequence
  Synthetic
GGAAAAAAAAASGSSGDGGSSGGSGGSGSSG

SEQ ID NO: 19 - protein, 31 aa, Artificial Sequence
  Synthetic
GGDDDDDDDDDSGSSGDGGSSGGSGGSGSSG

SEQ ID NO: 20 - protein, 31 aa, Artificial Sequence
  Synthetic
GGEEEEEEEEESGSSGDGGSSGGSGGSGSSG

SEQ ID NO: 21 - protein, 31 aa, Artificial Sequence
  Synthetic
GGGGGGGGGGGSGSSGDGGSSGGSGGSGSSG

SEQ ID NO: 22 - protein, 31 aa, Artificial Sequence
  Synthetic
GGHHHHHHHHHSGSSGDGGSSGGSGGSGSSG

SEQ ID NO: 23 - protein, 31 aa, Artificial Sequence
  Synthetic
GGMMMMMMMMMSGSSGDGGSSGGSGGSGSSG

SEQ ID NO: 24 - protein, 31 aa, Artificial Sequence
  Synthetic
GGNNNNNNNNNSGSSGDGGSSGGSGGSGSSG

SEQ ID NO: 25 - protein, 31 aa, Artificial Sequence
  Synthetic
GGPPPPPPPPPSGSSGDGGSSGGSGGSGSSG

SEQ ID NO: 26 - protein, 31 aa, Artificial Sequence
  Synthetic
GGQQQQQQQQQSGSSGDGGSSGGSGGSGSSG

SEQ ID NO: 27 - protein, 31 aa, Artificial Sequence
  Synthetic
GGRRRRRRRRRSGSSGDGGSSGGSGGSGSSG

SEQ ID NO: 28 - protein, 31 aa, Artificial Sequence
  Synthetic
GGSSSSSSSSSSGSSGDGGSSGGSGGSGSSG

SEQ ID NO: 29 - protein, 31 aa, Artificial Sequence
  Synthetic
GGTTTTTTTTTSGSSGDGGSSGGSGGSGSSG

SEQ ID NO: 30 - protein, 31 aa, Artificial Sequence
  Synthetic
GGRRGSYYSGGSGSSGDGGSSGGSGGSGSSG

SEQ ID NO: 31 - protein, 31 aa, Artificial Sequence
  Synthetic
GGRRGEYYSGGSGSSGDGGSSGGSGGSGSSG

SEQ ID NO: 32 - protein, 201 aa, Escherichia coli
MTMTRLKISKTLLAVMLTSAVATGSAYAENNAQTTNESAGQKVDSSMNKVGNFMDDSAITAKVKAALVDHDNIKSTDISVKTDQKVVTLSGFVESQAQAEEAVKVAKGVEGVTSVSDKLHVRDAKEGSVKGYAGDTATTSEIKAKLLADDIVPSRHVKVETTDGVVQLSGTVDSQAQSDRAESIAKAVDGVKSVKNDLKTK

SEQ ID NO: 33 - protein, 14 aa, Artificial Sequence
  Synthetic
MGHHHHHHHHHHGS

SEQ ID NO: 34 - protein, 96 aa, Artificial Sequence
  Synthetic
LQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQI

SEQ ID NO: 35 - protein, 47 aa, Artificial Sequence
  Synthetic
DGGSSGGSGGDGSSGDGGSDGDSDGSDGDGDSDGDDGGDDEDDGSDD

SEQ ID NO: 36 - protein, 118 aa, Escherichia coli
MKKRGAFLGLLLVSACASVFAANNETSKSVTFPKCEDLDAAGIAASVKRDYQQNRVARWADDQKIVGQADPVAWVSLQDIQGKDDKWSVPLTVRGKSADIHYQVSVDCKAGMAEYQRR

(19) United States
(12) Patent Application Publication
(10) Pub. No.: US 2021/0340192 A1
(43) Pub. Date: Nov. 4, 2021

(54) REPORTER CONSTRUCTS FOR NANOPORE-BASED DETECTION OF BIOLOGICAL ACTIVITY

(71) Applicant: UNIVERSITY OF WASHINGTON, Seattle, WA (US)

(72) Inventor: Jeffrey Matthew NIVALA, Seattle, WA (US)

(73) Assignee: UNIVERSITY OF WASHINGTON, Seattle, WA (US)

(21) Appl. No.: 17/283,007

(22) PCT Filed: Oct. 4, 2019
(86) PCT No.: PCT/US2019/054877

§ 371 Date: Apr. 5, 2021

Related U.S. Application Data
(60) Provisional application No. 62/741,670, filed on Oct. 5, 2018.

(51) Int. Cl. C07K 14/245; G01N 33/543

(52) U.S. Cl. CPC C07K 14/245; C07K 2319/035; G01N 33/54366

(57) ABSTRACT

The disclosure provides fusion reporter protein constructs and related compositions, systems, and methods for nanopore-based detection biological activity. In one aspect, the disclosure provides a fusion reporter protein comprising, in order: a blocking domain with a stably folded tertiary structure; a flexible analyte domain; and a flexible tail domain, wherein the flexible tail domain has a net negative charge. The disclosure also provides nucleic acid constructs encoding the disclosed fusion reporter protein, and vectors and cells comprising the nucleic acids. Also provided are nanopore-based systems and methods for using the disclosed fusion reporter protein constructs to detect and characterize biological activity.

### CROSS-REFERENCE TO RELATED APPLICATION

This application claims the benefit of U.S. Provisional Patent Application No. 62/741,670, filed Oct. 5, 2018, which is incorporated herein by reference in its entirety.

### STATEMENT OF GOVERNMENT LICENSE RIGHTS

This invention was made with Government support under 1841188 awarded by the National Science Foundation. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 70215_Sequence listing_ST25.txt. The text file is 19 KB; was created on Oct. 4, 2019; and is being submitted via EFS-Web with the filing of the specification.

### BACKGROUND

Reporter systems are essential for assaying the transcriptional and post-translational regulation of gene expression in biological systems. For nearly four decades, reporter proteins have been used to track such biological activities as genetic regulation. While several different reporter strategies have been developed over this period, the typical number of uniquely addressable reporters that can be used together while sharing a common readout is small. This limitation is primarily due to the optical nature of traditional reporters, such as fluorescent protein variants, which have overlapping spectral properties that make simultaneous measurement of unique genetic elements difficult. The ability to increase the ability to multiplex genetically-encoded protein reporters would enable more comprehensive and scalable monitoring of complex biological systems, enabling, for instance, high-dimensional phenotyping. This is particularly important for synthetic biology, in which scalable reporter systems are needed to keep pace with the complexity that biological systems can now be engineered in applications such as whole-cell biosensing and genetic circuit design. RNA-Seq is highly multiplexed approach that employs next-generation sequencing (NGS) to determine the presence and quantity of RNA gene transcripts in a biological sample to provide a snapshot of the cellular transcriptome. However, RNA templates are particularly susceptible to degradation during sample preparation, thus requiring additional steps to avoid skewing the results due to sample contamination. Furthermore, monitoring biological activity at the transcriptional level cannot address post-translational modification and regulation, thus providing an incomplete reflection of biological regulation in the system.

Accordingly, despite the advances in the art there remains a need for facile and robust approaches to monitoring protein expression, regulation modification, and in a manner that can be readily multiplexed to address highly complex systems. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a fusion reporter protein. The fusion reporter protein comprises, in order, a blocking domain with a stably folded tertiary structure, a flexible analyte domain, and a flexible tail domain, wherein the flexible tail domain has a net negative charge. In some embodiments, the flexible tail domain is configured to initiate translocation of the fusion reporting protein through a nanopore tunnel. In some embodiments, the blocking domain is configured to have the diameter exceeding a diameter of the nanopore total thereby preventing further translocation of the reporter protein through the nanopore tunnel when the blocking domain comes into contact with the nanopore.

In another aspect, the disclosure provides a nucleic acid comprising a sequence encoding the fusion reporter protein described herein. In some embodiments the nucleic acid further comprises a promoter or enhancer element operatively linked to the sequence encoding the fusion reporter protein.

In another aspect, the disclosure provides a vector comprising the nucleic acid described herein.

In another aspect the disclosure provides a cell comprising the nucleic acid and/or the vector described herein.

In another aspect, the disclosure provides a system. The system comprises:

a nanopore disposed in a barrier defining a cis side and a trans side, wherein the cis side comprises a first conductive liquid medium and the trans side comprises a second conductive liquid medium, and wherein the nanopore comprises a tunnel that provides liquid communication between the cis side and the trans side;

a data acquisition device operable to detect an ion current through the nanopore; and

a fusion reporter protein as described herein in the first liquid medium, wherein a diameter of the blocking domain of the reporter protein exceeds a diameter of the nanopore tunnel at its narrowest point.

In another aspect, the disclosure provides a method of detecting or characterizing biological activity of a biological system. The method comprises use of a nanopore system that comprises a nanopore disposed in a barrier defining a cis side and a trans side, wherein the cis side comprises a first conductive liquid medium and the trans side comprises a second conductive liquid medium, and wherein the nanopore comprises a tunnel that provides liquid communication between the cis side and the trans side. The method comprises:

providing a fusion reporter protein as described herein into the first conductive liquid medium of the cis side of the nanopore system;

initiating translocation of the flexible tail domain of the fusion reporter protein through the nanopore tunnel, wherein the blocking domain of the fusion reporter protein has a diameter that exceeds the diameter of the nanopore tunnel at its narrowest point;

measuring an ion current between the first conductive liquid medium and the second conductive liquid medium when the flexible analyte domain of the fusion reporter protein is in the tunnel of the nanopore; and detecting an ion current pattern associated with a structural characteristic of the flexible analyte domain of the fusion reporter protein.

The biological system can be, for example, one or more cells, or a cell free environment such as a cell lysate or artificial mixture that contains potentially active enzymes, and the like. The fusion reporter protein can be expressed or potentially modified in the biological system and then subjected to analysis in a nanopore system.

The method can be scaled-of and/or multiplexed and performed for the plurality of different fusion reporter proteins at the same time in the same reaction.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1F illustrate exemplary design and implementation of the disclosed Nanopore protein Tags Engineered as Reporters (NanoporeTERs or NTERs). FIG. 1A is a schematic design of an engineered gene encoding a NanoporeTER (NTER). The following exemplary domains are indicated: OsmY, which promotes extracellular secretion of the reporter protein in E. coli; Smt3, a folded domain that stalls translocation of the protein through the pore and facilitates a “static read” of the NTER barcode within the nanopore sensor; barcode (BC), which is a region of the protein that is held within the sensitive region of the nanopore lumen upon which the changes to the barcode sequence manifest changes to the nanopore ionic current signal; polyGSD tail, which is a long, flexible, negatively charged C-terminal domain promotes electrophoretic capture of the NTER into the nanopore under an applied voltage. FIG. 1B is a cartoon illustration of a NanoporeTER captured within a nanopore. FIG. 1C schematically illustrates that NanoporeTERs facilitate multiplexed readout of protein expression, with the potential to report on multiple outputs within a single strain (top), or report of expression across multiple strain types in a one-pot mix (bottom). FIG. 1D schematically illustrates an embodiment where secretion of the NanoporeTERs into the extracellular medium eliminates the need for any sample preparation prior to loading into the nanopore sensor array flow cell. FIG. 1E graphically illustrates an example of raw nanopore data generated from a single nanopore showing repeated captures and ejections events of an exemplary NanoporeTER, NTERY00. FIG. 1F graphically illustrates in exemplary concentration titration curve showing the relationship between NanoporeTER concentration within a flow cell versus the average time between captures or “reads.”

FIGS. 2A-2H illustrate mapping the NanoporeTER sequence and nanopore signal space on a MinION® device, according to an embodiment of the disclosure. FIG. 1A is a schematic of NTER Nos. 00-15 mutant sequences in which a sliding block of three tyrosine mutations was introduced along the NanoporeTER polyGSD barcode and tail region to map the NTER's nanopore-sensitive region and define the potential barcode sequence space. It is noted that each sequence has only a single aspartate residue at position 15. FIG. 2B is a violin plot showing the median ionic current level (normalized to the open pore level) of the nanopore capture state for NTER Nos. 00-15. Each NTER distribution is composed of several thousand single-molecule measurements. The introduction of the three tyrosine block (YYY), reduces the ionic current level in a position-dependent manner for positions 01-08. The median current level returns to the baseline (NTER 00) level starting at position 9 and through position 15, supporting a model in which the first 17 amino acids of the polyGSD tail contribute to the observed NTER ionic current signature, and defining the NTER barcode region. Each NTER distribution is composed of several thousand single-molecule measurements. FIG. 1C is a structural model of the NTER position within the nanopore during a read (capture event). A heat map displaying the relative change to specific signal features (median, standard deviation, minimum, and maximum) is projected onto the NTER tail residue positions (1-20) that were mutated in NTER Nos. 00-15, showing the relative magnitude of effect tyrosine mutations at each residue have on the NTER's nanopore ionic current signal. FIG. 1D graphically illustrates t-SNE plot clustering NTER reads (each read is represented as a single point) based on ionic current signal features (mean, std, min, max, median), and colored by the NTER's barcode identity (Y00-08). n=˜4000 events per barcode class. FIG. 1E is a violin plot showing the median ionic current level (normalized to the open pore level) of the nanopore capture state for amino acid homopolymer NTERs alanine (A), aspartate (D), glutamate (E), glycine (G), histidine (H), methionine (M), asparagine (N), proline (P), glutamine (Q), arginine (R), serine (S), and threonine (T). Each NTER distribution is composed of several thousand single-molecule measurements. FIG. 1F is a scatter plot showing the relationship between amino acid solvent accessible surface area (SASA) versus the respective amino acid homopolymer NTER mutant's median ionic current level (normalized to the open pore level). FIG. 1G is a scatter plot showing the relationship between amino acid helical propensity versus the respective amino acid homopolymer NTER mutant's median ionic current level (normalized to the open pore level). FIG. 1H is a kernel density plot comparing the ionic current median (normalized to the open pore level) of reads generated by an NTER containing a PKA phosphorylation motif (RRGSY) within its barcode region to those with a phosphomimetic mutation (RRGEY). Each NTER distribution is composed of several thousand single-molecule measurements.

FIGS. 3A-3D illustrate classification and multiplexed detection of NanoporeTER expression levels with a MinION. FIG. 3A illustrates exemplary raw ionic current data was classified using either a set of engineered features (mean, std, min, max, and median) or the unprocessed signal directly, and input into either a Random Forest or Convolutional Neural Network classifier, respectively. FIG. 3B illustrates exemplary confusion matrices showing the Random Forest test set classification accuracies on models using different combination of NTER barcodes. Top left: NTER Nos. 00-08. Bottom left: amino acid homopolymer mutants A, D, E, G, H, M, N, P, Q, R, S, and T. Right: Both the NTER Nos. 00-08 and amino acid homopolymer mutants. FIG. 1C provides a schematic diagram showing the gene construct used for controllable NTER expression (left). IPTG is used to induce NTER expression (“ON”), while glucose inhibits expression (“OFF”). The diagram and bar plot on the right shows the results of a mixed culture experiments in which NTER expression was induced for NTER Nos. Y02 and Y04, and inhibited for NTER Nos. Y00, Y02, and Y08. NTER Nos. Y01, Y03, Y05, and Y07 were held out of the experiment as negative controls. Plot shows the total number of reads classified as each NTER barcode during MinION® analysis. FIG. 1D is a line plot showing a time course of NTER expression levels as determined by the rate of classified reads (reads/pore/min) for each NTER barcode. NTER Y06 was induced, while NTER Y02 was inhibited. The other NTERs were held out as negative controls and show false-positive classification rates. Three replicates for each condition are plotted.

FIGS. 4A and 4B illustrate that NanoporeTERs that include secretion domains are secreted into the extracellular medium. FIG. 4A illustrates a cartoon schematic of the NTER design, including an OsmY domain for secretion in E. coli. The lower panel illustrates SDS-PAGE analysis of overnight culture of an E. coli strain transformed with a plasmid expressing NTER00 (expected MW is 40.2 kilodaltons). Lanes: 1, Ladder; 2, raw whole culture (cells and growth medium); 3, cell pellet resuspended in water following centrifugation; 4, Growth medium supernatant following centrifugation. Secreted NTER is indicated. FIG. 4A illustrates a cartoon schematic of the NTER design, including an IFNα2 domain for secretion in human cell lines. Lane 1 is a letter in the lane 2 is the growth medium supernatant following centrifugation. Secreted NTER cells from HEK293 cells is indicated. Additional protein bands are confirmed as being from the growth media.

FIG. 5 is a series of violin plots showing the ionic current level signal characteristics (mean, std, min, and max; all normalized to the open pore level) of the nanopore capture state for NTER Nos. 00-15. Each NTER distribution is composed of several thousand single-molecule measurements.

FIG. 6 is a series of violin plots showing the ionic current level signal characteristics (mean, std, min, and max; all normalized to the open pore level) of the nanopore capture state for the amino acid homopolymer mutants. Each NTER distribution is composed of several thousand single-molecule measurements.

FIGS. 7A-7C illustrate exemplary use of NTER constructs as reporters of post-translation modifications. FIG. 7A schematically illustrates an exemplary NTER held statically in the nanopore by the folded domain. The analyte domain occupies the narrowest portion of the nanopore tunnel. The sequence of the analyte domain contains a casein kinase II (CKII) domain based on the motive SXXD, which can result in phosphorylation of the serine of the motif. FIG. 7B graphically illustrates the kernel density versus nanopore signal mean for NTERs with the CKII domain that were previously incubated with a kinase for 0, 1 hour, and 12 hours. The peaks relating to detection of phosphorylated and unmodified NTERs are indicated. FIG. 7C graphically illustrates the proportion of signal events (i.e., for unmodified or phosphorylated NTERs) for the different kinase incubation times of the NTERs containing the CKII domain.

### DETAILED DESCRIPTION

Genetically encoded reporter proteins are a cornerstone of molecular biology. While they are widely used to measure many biological activities, the current number of uniquely addressable reporters that can be used together for one-pot multiplexed tracking is small due to overlapping detection channels, such as fluorescence. This disclosure provides protein reporter constructs to monitor gene expression and regulation using nanopore based systems that permit high levels of potential multiplexing without resulting in overlapping detection signals. As described in more detail below, an expanded library of orthogonally-barcoded Nanopore-addressable protein Tags Engineered as Reporters (“NanoporeTERs” or “NTERs”; also referred to as “fusion reporter proteins”) was constructed. The NanoporeTER constructs were demonstrated to be read and demuxed by nanopore sensors at the single-molecule level. For proof of concept, a commercially available nanopore sensor array platform typically used for real-time DNA and RNA sequencing (e.g., Oxford Nanopore Technologies' (ONT's) MinION®) was adapted to detection of different NanoporeTER constructs. Direct detection of NanoporeTER expression levels from unprocessed bacterial culture with no specialized sample preparation was demonstrated. The reporter constructs, and related methods and systems, described herein provide for a highly flexible approach to detect and characterize biological activities, such as activity of promoters/enhancers and corresponding transcription factors, and activity of enzymes that can modify proteins in particular target sequences. Furthermore, the disclosed results establish that this new class of reporter proteins can provide for highly multiplexed, real-time tracking of the biological activity in one pot reactions using nascent nanopore sensor technology.

Fusion Reporter Protein

In view of the foregoing, in one aspect the disclosure provides a fusion reporter protein comprising, in order: a blocking domain with a stably folded tertiary structure, a flexible analyte domain, and a flexible tail domain, wherein the flexible tail domain has a net negative charge.

The order of the blocking domain, the flexible analyte domain, and the flexible tail domain can be from a relative N-terminal position within the fusion reporter protein to a relative C-terminal position within the fusion reporter protein. Alternatively, the order of the blocking domain, the flexible analyte domain, and the flexible tail domain can be from a relative C-terminal position within the fusion reporter protein to a relative N-terminal position within the fusion reporter protein. The terms “relative N-terminal position” and “relative C-terminal position” do not require that the respective domains are at the terminal ends of the fusion protein, but rather they indicate the positioning of the domains along the linear fusion reporter protein sequence with respect to their relative proximity to terminal ends. Ultimately, regardless of the order of the domains, the flexible analyte domain is disposed between the blocking domain and the flexible tail domain. Any two or all three domains can be contiguous, or can be separated by intervening linker domains. The linker domains are typically short amino acid sequences that do not confer functionality other than inserting space between the domains. In some embodiments all three of the indicated domains are positioned contiguously.

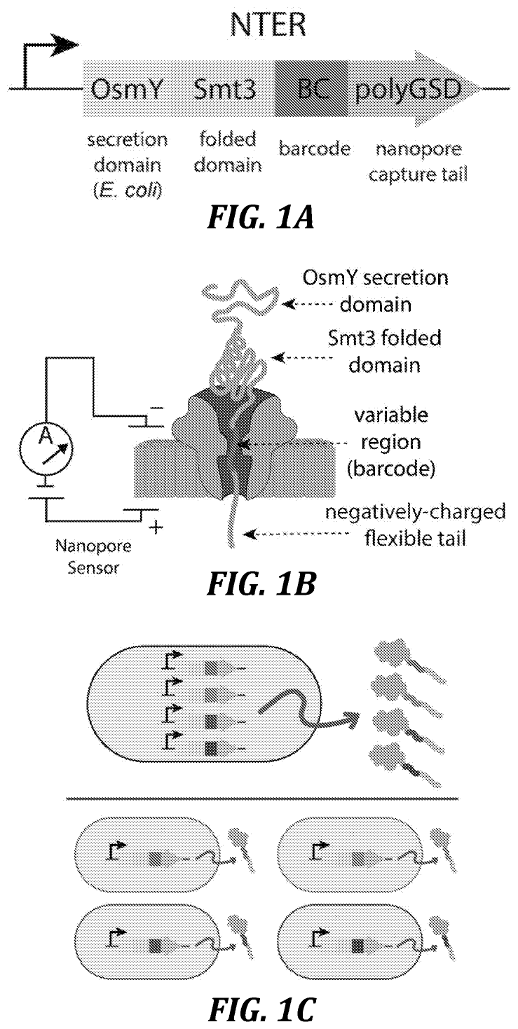

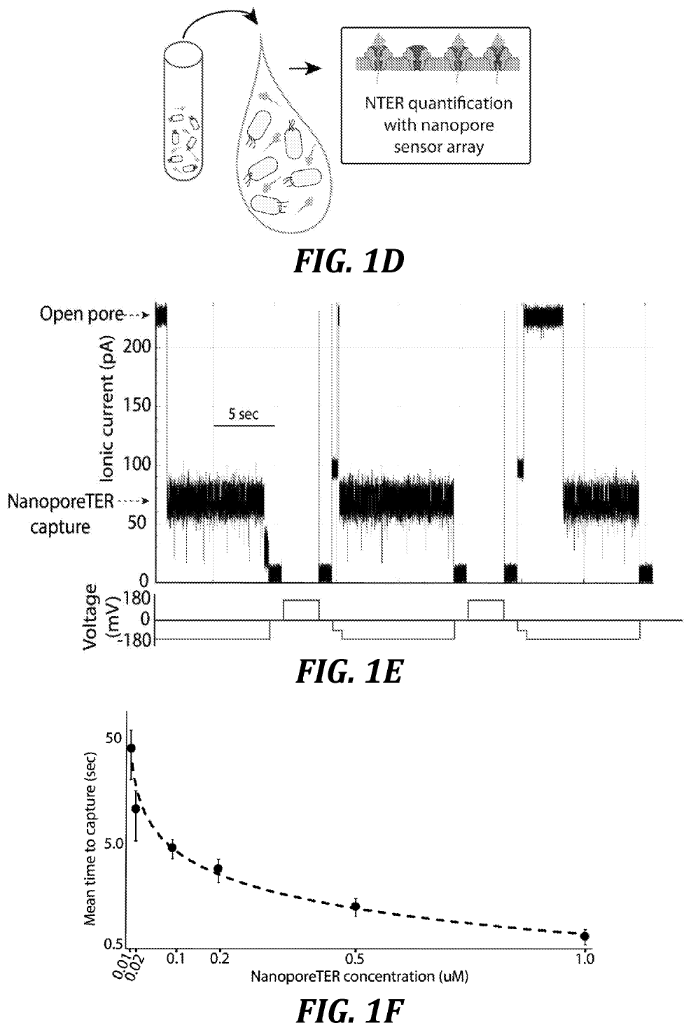

The blocking domain and the flexible tail domain are each configured to provide the functionality of the fusion reporter protein with respect to a nanopore. Nanopores and systems incorporating nanopores for polymer analysis are described in more detail below. With respect to the fusion reporter protein, in some embodiments the flexible tail domain is configured to initiate translocation of the fusion reporting protein through a nanopore tunnel. Translocation proceeds with the flexible tail domain and followed by the flexible analyte domain. The blocking domain is configured to have a diameter exceeding a diameter of the nanopore total, thereby preventing further translocation of the reporter protein through the nanopore tunnel when the blocking domain comes into contact with the nanopore. These configured functionalities of the flexible tail domain and the blocking domain are illustrated for a specific embodiment in FIG. 1B, which illustrates a negatively charged flexible tail domain having interacted and translocated through the tunnel of a nanopore. As the linear polypeptide structure of the fusion reporter protein translocates in a linear fashion through the nanopore, the blocking domain (illustrated here as “Smt3 folded domain”) is eventually pulled against the outer rim of the nanopore. As illustrated, the blocking domain has a diameter that exceeds the diameter of the internal tunnel of the nanopore. Therefore, progress of translocation is halted with the blocking domain is held against the relatively narrow opening of the nanopore. This this configuration leaves the analyte domain (illustrated here as “variable region (barcode)”) in interior of the nanopore, with the negatively charged flexible tail domain having translocated to the other side.

Accordingly, the blocking domain has a minimal diameter that exceeds the diameter of the nanopore to prevent translocation. This minimal diameter can be dictated by the corresponding diameter of the nanopore to which fusion reporter protein may be applied in an essay (see description of exemplary nanopores below). In some embodiments, the blocking domain has a folded tertiary structure with a diameter greater than about 1.5 nm. For example, the blocking domain can have a folded tertiary structure with a diameter greater than about 1.5 nm, about 1.75 nm, about 2.0 nm, about 2.25 nm, about 2.5 nm, about 2.75 nm, about 3.0 nm, or greater. It will be apparent to practitioners in the art that there is no theoretical upper bound to the smallest diameter of the blocking domain's tertiary structure. The required functionality is to simply be larger than the diameter of the nanopore tunnel such that the blocking domain prevents further translocation of the fusion reporter protein through the nanopore. However, it may be advantageous to retain a relatively small size for the blocking domain for ease of production and expression of the fusion reporter protein within a cell, and to avoid interference with the functionalities of the flexible analyte domain and flexible tail domain.

In some embodiments, the primary sequence of the blocking domain consists of about 40 to about 500 amino acids. In some embodiments, the primary sequence of the blocking domain consists of about 40 to about 400 amino acids; about 50 to about 350 amino acids; about 50 to about 300 amino acids; about 50 to about 250 amino acids; about 50 to about 200 amino acids; about 75 to about 350 amino acids; about 75 to about 300 amino acids; about 75 to about 250 amino acids; about 75 to about 200 amino acids; about 100 to about 350 amino acids; about 100 to about 300 amino acids; about 100 to about 250 amino acids; about 100 to about 200 amino acids; about 125 to about 350 amino acids; about 125 to about 300 amino acids; about 125 to about 250 amino acids; and about 125 to about 200 amino acids. For example, the sequence of the blocking domain can consist of about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, and about 500 amino acids.

The blocking domain has a folded tertiary structure that is stable. In this context, the term “stable” indicates that the blocking domain maintains its tertiary structure, i.e. resist denaturing, under conditions that would be typical for nanopore analysis in a nanopore system. For example, as described in more detail below, nanopore-based assays were performed by applying electrical current in conductive liquid media to drive the interaction of the fusion reporter protein with a nanopore. Accordingly, the stability of the blocking domain can be mechanical in the sense that it resists being unfolded when subjected to a pulling force when the blocking domain is pulled up against the opening of the nanopore. Additionally, the stability is chemical in the sense that it resists denaturing in the presence of a chemical environment, such that it includes ionic conditions, urea, and the like. Furthermore, the tertiary structure of the blocking domain must be sufficiently stable in the presence of an electrical field. In some embodiments, the tertiary structure of the blocking domain remains stable at 37° C. in conditions comprising at least about 500 mM KCl. In some embodiments the blocking domain contains one or more disulfide bonds that contribute to the stability of the tertiary structure.

Additionally, in some embodiments the blocking domain is configured to retain high solubility in salt conditions, which are typical of the nanopore experiments. Retaining solubility facilitates an efficient assay and avoids fusion reporter protein analytes from precipitating out of solution.

For purposes of illustration, non-limiting embodiments of blocking domains encompassed by the disclosure include blocking domains that comprise small ubiquitin related modifier (SUMO)-like domains or titin protein domains. SUMO proteins tend to be small, such as about 100 amino acids in length and about 12 kDa in mass. In one embodiment, the blocking domain comprises the SUMO-like protein Smt3. Sequence for Smt3 protein is set forth in SEQ ID NO:34. Thus, in some embodiments the blocking domain comprises an amino acid sequence with at least about 80%, at least about 85%, at least about 90%, at least about 95%, at least about 98%, at least about 99% sequence identity to SEQ ID NO:34. As referred to herein, a titin protein domain is a discrete subdomain of the large titin protein found in striated muscle. The native titin protein comprises numerous (e.g., 244) individual, discrete titin protein domains, each of which maintains a highly stable folded structure. These individual titin domains are connected within the native protein by unstructured peptide sequences. See, e.g., Abolbashari, M. H. and S. Ameli, “Mechanical unfolding of titin 127 domain: Nanoscale simulation of mechanical properties based on virial theorem via steered molecular dynamics technique,” Scientia Iranica, 19(6):1526-1533:2012 (2012), incorporated herein by reference in its entirety. The present disclosure encompasses embodiments wherein the blocking domain comprises a single titin (sub)domain.

As indicated above, the flexible analyte domain is disposed between the blocking domain and the flexible tail domain. The flexible analyte domain is configured to translocate through the opening into the interior of a nanopore. Due to the blocking action of the blocking domain, the flexible analyte domain can be held static in the narrowest section (i.e., “construction zone”) of the nanopore tunnel, and thereby influence current passing through the tunnel to provide detectable signals in a nanopore system (this is addressed below in more detail). Accordingly, the analyte domain is flexible to facilitate passage into the nanopore. Some embodiments, the flexible analyte domain lacks tertiary structure. The lack of folding prevents formation of configurations whereby the domain might be prevented from passage to the nanopore, such as exhibited by the blocking domain. In other embodiments, the flexible analyte domain also lacks secondary structure; however, this is not a requirement for functionality as secondary helix structures could still pass through a nanopore opening.

The flexible analyte domain can contain as few as a single amino acid in its sequence. In some embodiments the analyte domain comprises about 1 amino acid to about 30 amino acids, such as about 1 amino acid to about 25 amino acids, about 2 amino acids to about 25 amino acids, about 4 amino acids to about 25 amino acids, about 5 amino acids to about 25 amino acids, about 10 amino acids to about 25 amino acids, about 12 amino acids to about 25 amino acids, about 15 amino acids to about 25, about 1 amino acid to about 20 amino acids, about 2 amino acids to about 20 amino acids, about 4 amino acids to about 20 amino acids, about 5 amino acids to about 20 amino acids, about 10 amino acids to about 20 amino acids, about 12 amino acids to about 20 amino acids, about 15 amino acids to about 20 amino acids. In some embodiments, the flexible analyte domain comprises or consists of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 amino acids.

In some embodiments the flexible analyte domain comprises an amino acid sequence containing a uniquely identifiable barcode. As used herein, the term “identifiable barcode” refers to the ability to detect and differentiate a particular unique barcode sequence in relation to different barcode sequences in other analyte domains using, e.g., a nanopore detection platform. As illustrated in, e.g., FIG. 1B, the flexible analyte domain can be held static in the construction zone of the nanopore interior, whereby the specific structure (i.e., sequence) can influence the detectable current passing through the nanopore. In some embodiments, in the context of a plurality of fusion reporter proteins, the barcode sequence of the flexible analyte domain can be referred to as being degenerate. As a result, each individual flexible analyte domain in the plurality of flexible analyte domains has a different barcode sequence that is unique to each fusion reporter protein in the plurality and which is uniquely identifiable in a nanopore system. As described in more detail below, it was determined that as few as a single amino acid difference in the analyte domain sequences of different fusion reporter proteins can be distinguished (i.e., identified) in a nanopore system.

In other embodiments, the flexible analyte domain has an amino acid sequence that contains a target sequence for a post-translation modification. The term “post translation modification” encompasses any modification that can be imposed on a peptide or protein. Exemplary, nonlimiting modifications encompassed by the disclosure include phosphorylation, methylation, glycosylation, acetylation, lipidation, nitrosylation, and the like, although additional post-translation modifications are known in the art also encompassed by the present disclosure. Target sequences for such post-translation modifications are known and are encompassed by the present disclosure. For example, SEQ ID NO:30 is an exemplary analyte domain sequence that comprises a target for protein kinase A (PKA) phosphorylation motif (see, e.g., Taylor, S. S., et al., “PKA: A portrait of protein kinase dynamics,” Biochimica et Biophysica Acta—Proteins and Proteomics 1697(1-2):259-269 (2004), incorporated herein by reference in its entirety). With such target sequence incorporated into the analyte domain, the fusion reporter protein can be acid in a nanopore system for the presence of a post translation modification.

As indicated above, the flexible tail domain is configured to provide functionality to the reporter protein, namely, it is configured to facilitate initial interaction with a nanopore and initiate translocation of the linear polypeptide molecule through the nanopore until such a time that the blocking domain prevents further translocation. To maximize the likelihood of interaction with the nanopore in initiation of translocation through the nanopore, the flexible tail domain preferably lacks tertiary structure. In some embodiments the flexible tail domain also lacks secondary structure, although this is not necessary for functionality as a helix secondary structure can hypothetically thread through a nanopore tunnel.

The flexible tail domain can be relatively short in sequence so long as it is able to interact with a nanopore. In some embodiments the flexible tail domain comprises at least about 15 amino acids, at least about 20 amino acids, at least about 25 amino acids, at least about 30 amino acids, at least about 35 amino acids, at least about 40 amino acids, at least about 45 amino acids, at least about 50 amino acids, at least about 55 amino acids, or more amino acids. In some embodiments, the flexible tail domain comprises between about 20 and about 150 amino acids, such as between about 20 and about 100 amino acids, between about 25 and about 90 amino acids, between about 30 and about 90 amino acids, and between about 40 and about 80 amino acids. In some embodiments, the flexible tail domain comprises or consists of about 20, about 21, about 22, about 23, about 24, about 25, about 30, about 35, about 40, about 45, about 50, about 55, about 60, about 65, about 70, about 75, about 80, about 85, about 90, about 95, about 100, about 110, about 120, about 130, about 140, about 150 amino acids.

As indicated above, the flexible tail domain has a net negative charge. The negative charge facilitates interaction with nanopores in current nanopore platforms that are presently used in DNA sequencing. Given the negative charge of DNA, the commonly used nanopores tend to have neutral or positive charges and utilize a voltage polarity that facilitates movement of the negatively charged DNA polymer through the nanopore. Thus, to facilitate operation with the same nanopore platform technologies, in some embodiments the flexible tail domain comprises one or more negatively charged amino acids, such as aspartic acid, and glutamic acid, in any combination or proportion. In additional embodiments, the flexible tail domain also comprises one or more of glycine in serine residues, in any combination or proportion. Glycine in serine residues can be included because they are relatively small residues and facilitate the flexibility of the flexible tail domain. In some embodiments, the flexible tail domain consists of, or consists essentially of, glycine residues, serine residues, aspartic acid residues, glutamic acid residues, or any combination thereof. As used in this context, the phrase “consists essentially of” indicates that the flexible tail domain can contain additional amino acid residues not listed here, but which do not substantially or significantly alter the net charge or flexible structure of the flexible tail domain.

While the above disclosure is presented generally in the context of having a flexible tail domain with the net negative charge, it will be appreciated that nanopore systems can be developed or modified to wherein the voltage polarity applied to the nanopore sensor is in the opposite direction, and/or the nanopore itself has a negative charge. Thus, the present disclosure also encompasses alternative embodiments wherein the flexible tail domain does not have a net negative charge, but rather can have neutral or positive charge incorporated therein to facilitate interaction with the nanopore in the presence of an appropriately configured voltage field. Amino acids residues such as arginine, lysine, and histidine are basic and, thus, can confer positive charge to the flexible tail domain.

In some embodiments, the fusion reporter protein further comprises a secretion domain. The secretion domain can be any secretion domain that facilitates transport of the translated fusion reporter protein to the exterior of a cell in which the fusion reporter protein is expressed. The secretion domain is typically positioned within the fusion reporter protein on the side of the blocking domain opposite the flexible analyte domain. Thus, in some embodiments the fusion reporter protein comprises, in order: the secretion domain, the blocking domain, the flexible analyte domain, and the flexible tail domain. As indicated above, this recited order can be in relative N-terminal to C-terminal order, or it can be in relative C-terminal to N-terminal order, so long as the particular secretion domain is functional on the N-terminus or C-terminus, respectively, of an expressed protein.

The secretion domain can be designed and selected based on the cell type in which the fusion reporter protein is expressed according to standard knowledge and skill of the art. In some embodiments the cell type of interest is a prokaryotic cell, such as bacteria. In a specific embodiment, the cell type of interest is E. coli, or any other bacterial cell amenable to serve as a gene expression platform. Secretion domains that are functional in prokaryotic cell expression systems are known and are encompassed by the present disclosure. In one embodiment, the secretion domain is an OsmY secretion domain. A representative sequence of the OsmY secretion domain is set forth herein as SEQ ID NO:32. Accordingly, in some embodiments the fusion reporter protein comprises a secretion domain (e.g., in a position on the N-terminal side of the blocking domain), wherein the secretion domain comprises an amino acid sequence with at least 80% sequence identity to the sequence of SEQ ID NO:32, or functional fragments thereof. In another embodiment, the secretion domain is a YebF secretion domain. A representative sequence of the YebF secretion domain is set forth herein as SEQ ID NO:36. Accordingly, in some embodiments the fusion reporter protein comprises a secretion domain (e.g., in a position N-terminal to the blocking domain), wherein the secretion domain comprises an amino acid sequence with at least 80% sequence identity to the sequence of SEQ ID NO:36, or functional fragments thereof. The term “functional fragment” refers to a subdomain or shorter sequence of the references sequence that retains functional activity for promoting secretion of the fusion protein containing a functional fragment.

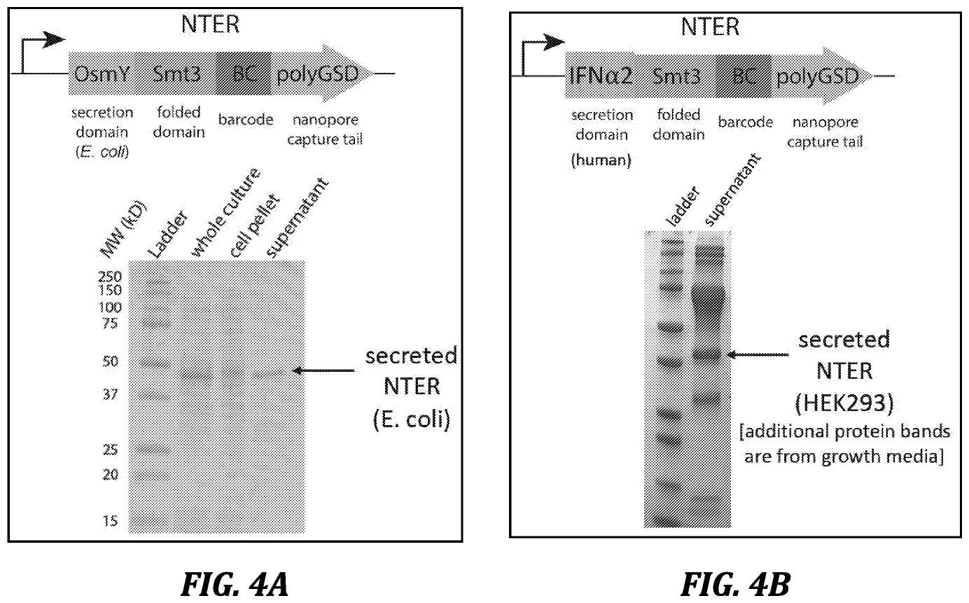

In other embodiments, the cell type of interest is a eukaryotic cell and, thus, the secretion domains are functional to facilitate secretion by a eukaryotic cell. Secretion domains that are functional in eukaryotic cell expression systems are known and are encompassed by the present disclosure. For example, as described in more detail below, FIG. 4A illustrates the successful use of IFNα2 as a secretion domain in eukaryotic cells (i.e., human HEK293 cells) to produce fusion reporter proteins. See also, e.g., Roman, R., et al., “Enhancing heterologous protein expression and secretion in HEK293 cells by means of combination of CMV promoter and IFNα2 signal peptide,” J. of Biotechnology, 239(10):57-60 (2016), incorporated herein by reference in its entirety.

Nucleic Acid and Related Constructs

In another aspect, the present disclosure also provides nucleic acid constructs that encode the fusion reporter proteins described herein. The nucleic acid construct can be DNA or RNA. In some embodiments the nucleic acid construct further comprises a promoter or enhancer element that is operatively linked to the sequence encoding the fusion reporter protein. As used herein, the term “operatively linked” indicates that the promoter or enhancer sequence and the nucleic acid encoding the fusion reporter protein are configured and positioned relative to each other a manner such that the promoter or enhancer can activate transcription of the encoding nucleic acid by the transcriptional machinery of the cell. The promoter or enhancer sequence can be selected and configured by person of ordinary skill in the art to promote expression of the fusion reporter protein in the cell of interest. In some embodiments, the particular promoter or enhancer sequence is chosen to ascertain whether it is functional, or to what degree it is functional, to promote expression within the cell type of interest.

In another aspect, the disclosure provides a vector comprising the nucleic acid described above. The vector can be any construct that facilitates the delivery of the nucleic acid to the target cell and/or expression of the nucleic acid within the cell. The vectors can be viral vectors, circular nucleic acid constructs (e.g., plasmids), or nanoparticles. In some embodiments the vectors further comprise elements that promote functionality, such as origins of replication and selection resistance.

In yet another aspect, the disclosure provides a cell comprising the nucleic acid encoding any fusion reporter protein described herein. In some embodiments the cell comprises a vector disclosed herein, wherein the vector comprising the nucleic acid encoding fusion reporter protein. In some embodiments the cell can be referred to as a target cell, which indicates that the focus of an assay is on the biological system and functionality of the target cell. To illustrate, a promoter may be incorporated into the nucleic acid expressing the fusion reporter protein for an assay to determine the functionality of the reporter protein in the target cell.

Systems

In another aspect, the disclosure provides a system comprising a nanopore and a fusion reporter protein as described herein.

In some embodiments, the system comprises:

a nanopore disposed in a barrier defining a cis side and a trans side, wherein the cis side comprises a first conductive liquid medium and the trans side comprises a second conductive liquid medium, and wherein the nanopore comprises a tunnel that provides liquid communication between the cis side and the trans side;

a data acquisition device operable to detect an ion current through the nanopore; and

a fusion reporter protein as described herein in the first liquid medium, wherein a diameter of the blocking domain of the reporter protein exceeds a diameter of the nanopore tunnel at its narrowest point.

Various aspects of the nanopore systems as employed in the present disclosure are described below.

Nanopore-based analysis methods have previously been investigated for the characterization of analytes that are passed through the nanopore. As described above, nanopore systems have been established specifically for the analysis of nucleic acid polymers, for example single-stranded DNA (“ssDNA”), which pass linearly through a nanoscopic opening of the nanopore while providing a signal, such as an electrical signal, that is influenced by the physical properties of the nucleotide subunits that reside in the close physical space of the nanopore tunnel at any given time. As described in more detail below, such extant and nascent nanopore systems can be co-opted for other polymer analyses, such as for linearized portions of the disclosed fusion reporter protein molecules.

The nanopore of the presently disclosed system optimally has a size or three-dimensional configuration that allows the flexible domains of the fusion reporter protein to pass through only in a sequential, single file order. Chemical and physical properties of each monomeric amino acid subunit that makes up the flexible domains of the reporter protein can influence electrical signals. Thus, the particular sequence, such as a barcode sequence in the flexible analyte domain, can result in a detectable signal characteristic of the analyte barcode as it passes through and/or resides within nanopore. Alternatively, the modification status of a target sequence within the analyte domain (e.g., methylated or not; phosphorylated or not) can result in the detectable signal to determine the presence or absence of the modification.

A “nanopore” specifically refers to a pore typically having a size of the order of a few nanometers that allows the passage of analyte polymers (such as polypeptide polymers) therethrough. Typically, nanopores encompassed by the present disclosure have an opening with a diameter at its most narrow point of about 0.3 nm to about 2 nm. Nanopores useful in the present disclosure include any pore capable of permitting the linear translocation of the fusion reporter protein, and more specifically the flexible domains of the fusion reporter protein which are linear and lack tertiary structure, through the nanopore.

Nanopores can be biological nanopores (e.g., proteinaceous nanopores), solid state nanopores, hybrid solid state protein nanopores, a biologically adapted solid state nanopore, a DNA origami nanopore, and the like.

In some embodiments, the nanopore comprises a protein, such as alpha-hemolysin, anthrax toxin and leukocidins, and outer membrane proteins/porins of bacteria such as Mycobacterium smegmatis porins (Msp), including MspA, outer membrane porins such as OmpF, OmpG, OmpATb, and the like, outer membrane phospholipase A and Neisseria autotransporter lipoprotein (NaIP), and lysenin, as described in U.S. Publication No. US2012/0055792, International PCT Publication Nos. WO2011/106459, WO2011/106456, WO2013/153359, and Manrao et al., “Reading DNA at single-nucleotide resolution with a mutant MspA nanopore and phi29 DNA polymerase,” Nat. Biotechnol. 30:349-353 (2012), each of which is incorporated herein by reference in its entirety. In other embodiments the protein nanopore is CsgG, ClyA, or aerolysin. Nanopores can also include alpha-helix bundle pores that comprise a barrel or channel that is formed from a-helices. Suitable α-helix bundle pores include, but are not limited to, inner membrane proteins and outer membrane proteins, such as WZA and ClyA toxin. In one embodiment, the protein nanopore is a heteroligomeric cationic selective channel from Nocardia faricinica formed by NfpA and NfpB subunits. The nanopore can also be a homolog or derivative of any nanopore described above. A “homolog,” as used herein, is a protein from another species that has a similar structure and evolutionary origin. By way of an example, homologs of wild-type MspA, such as MppA, PorM1, PorM2, and Mmcs4296, can serve as the nanopore in the disclosed system. Protein nanopores have the advantage that, as biomolecules, they self-assemble and are essentially identical to one another. In addition, it is possible to genetically engineer protein nanopores, thus creating a “derivative” of a nanopore that possesses various attributes. Such derivatives can result from substituting amino acid residues for amino acids with different charges, from the creation of a fusion protein. Thus, the protein nanopores can be wild-type or can be modified to contain at least one amino acid substitution, deletion, or addition. In some embodiments, the at least one amino acid substitution, deletion, or addition results in removal of a steric barrier to translocation of the flexible domains through the nanopore. In some embodiments, the at least one amino acid substitution, deletion, or addition results in a different net charge of the nanopore. In some embodiments, the difference in net charge increases the difference of net charge as compared to the first charged moiety of the polymer analyte. For example, if the first charged moiety has a net negative charge, the at least one amino acid substitution, deletion, or addition results in a nanopore that is less negatively charged. In some cases, the resulting net charge is negative (but less so), is neutral (where it was previously negative), is positive (where it was previously negative or neutral), or is more positive (where it was previously positive but less so). In some embodiments, the alteration of charges in the nanopore entrance rim or within the interior of the tunnel and/or constriction facilitate the entrance and interaction of the polymer with the nanopore tunnel.

In some embodiments, the nanopores can include or comprise DNA-based structures, such as generated by DNA origami techniques. For descriptions of DNA origami-based nanopores for analyte detection, see PCT Publication No. WO2013/083983, incorporated herein by reference.

Some nanopores can comprise a variably shaped tunnel component through which the flexible domains of the fusion reporter protein move. FIG. 1B provides a diagram that illustrates an exemplary nanopore configuration where the nanopore is disposed in a membrane. The membrane serves as a barrier between a top area and bottom area, and also referred to herein as a cis side and trans side. In the cis side, the nanopore has an outer entrance rim region provides a relatively wide opening into the tunnel through which the linear flexible tail domain has passed, followed by the flexible analyte domain (labeled as “variable region (barcode)”). The widest interior section of the tunnel is often referred to as the vestibule. In contrast, the narrowest portion of the interior tunnel is referred to as the constriction zone. The vestibule and a constriction zone together form the tunnel. In the illustrated nanopore the rim and vestibule together form a cone-shaped portion of the interior of the nanopore whose diameter generally decreases from one end to the other along a central axis, where the narrowest portion of the vestibule is connected to the constriction zone. The indicated flexible analyte domain is held static in the constriction zone. Stated otherwise, the vestibule of the illustrated nanopore can generally be visualized as “goblet-shaped.” Because the vestibule is goblet-shaped, the diameter changes along the path of a central axis, where the diameter is larger at one end than the opposite end. The diameter may range from about 2 nm to about 6 nm. Optionally, the diameter is about, at least about, or at most about 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 nm, or any range derivable therein. The length of the central axis may range from about 2 nm to about 6 nm. Optionally, the length is about, at least about, or at most about 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 nm, or any range derivable therein. When referring to “diameter” herein, one can determine a diameter by measuring center-to-center distances or atomic surface-to-surface distances.

The term “constriction zone” generally refers to the narrowest portion of the tunnel of the nanopore, in terms of diameter, that is connected to the vestibule. The length of the constriction zone can range, for example, from about 0.3 nm to about 20 nm. Optionally, the length is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. The diameter of the constriction zone can range from about 0.3 nm to about 5 nm. Optionally, the diameter is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. In other embodiment, such as those incorporating solid state pores, the range of dimension (length or diameter) can extend up to about 20 nm. For example, the constriction zone of a solid state nanopore is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, or 5 nm, or any range derivable therein. The constriction zone is generally the part of the nanopore structure where the presence of a polymer, such as the fusion reporter protein, can influence the ionic current from one side of the nanopore to the other side of the nanopore. In some instances, the term “constriction zone” is used in a functional context based on the obtained resolution of the nanopore and, thus, the term is not necessarily limited by any specific parameter of physical dimension. Depending on physical characteristics the nanopore and the overall system, the length (i.e., number of amino acid residues in a linear sequence) of the flexible analyte domain that influence a detectable and distinguishable signal from a nanopore system can vary.

In some embodiments, the nanopore can be a solid state nanopore. A solid-state layer is not of biological origin. In other words, a solid-state layer is not derived from or isolated from a biological environment such as an organism or cell, or a synthetically manufactured version of a biologically available structure. Solid state nanopores can be produced as described in U.S. Pat. Nos. 7,258,838 and 7,504,058, incorporated herein by reference in their entireties. Briefly, solid state layers can be formed from both organic and inorganic materials including, but not limited to, microelectronic materials, insulating materials such as Si3N4, Al2O3, and SiO, organic and inorganic polymers such as polyamide, plastics such as Teflon®, or elastomers such as two-component addition-cure silicone rubber, and glasses. The solid-state layer may be formed from graphene. Suitable graphene layers are disclosed in WO 20091035647 and WO 20111046706. Solid state nanopores have the advantage that they are more robust and stable. Furthermore, solid state nanopores can in some cases be multiplexed and batch fabricated in an efficient and cost-effective manner. Finally, they might be combined with micro-electronic fabrication technology. In some embodiments, the nanopore comprises a hybrid protein/solid state nanopore in which a nanopore protein is incorporated into a solid state nanopore. In some embodiments, the nanopore is a biologically adapted solid-state pore.

In some cases, the nanopore is disposed within a membrane, thin film, layer, or bilayer. For example, biological (e.g., proteinaceous) nanopores can be inserted into an amphiphilic layer such as a biological membrane, for example, a lipid bilayer. An amphiphilic layer is a layer formed from amphiphilic molecules, such as phospholipids, which have both hydrophilic and lipophilic properties. The amphiphilic layer can be a monolayer or a bilayer. The amphiphilic layer may be a co-block polymer. Alternatively, a biological pore may be inserted into a solid-state layer.

The membrane, thin film, layer, or bilayer typically separates a first conductive liquid medium and a second conductive liquid medium to provide a nonconductive barrier between the first conductive liquid medium and the second conductive liquid medium. The nanopore, thus, provides liquid communication between the first and second conductive liquid media through its internal tunnel. In some embodiments, the pore provides the only liquid communication between the first and second conductive liquid media. The conductive liquid media typically comprises electrolytes or ions that can flow from the first conductive liquid medium to the second conductive liquid medium through the interior of the nanopore. Liquids employable in methods described herein are well-known in the art. Descriptions and examples of such media, including conductive liquid media, are provided in U.S. Pat. No. 7,189,503, for example, which is incorporated herein by reference in its entirety. The first and second liquid media may be the same or different, and either one or both may comprise one or more of a salt, a detergent, or a buffer. Indeed, any liquid media described herein may comprise one or more of a salt, a detergent, or a buffer. Additionally, any liquid medium described herein may comprise a viscosity altering substance or a velocity altering substance.

In some cases, the first and second conductive liquid media located on either side of the nanopore are referred to as being on the cis and trans regions, where the fusion reporter protein is provided in the cis region. In some embodiments, the nanopore or portion thereof in contact with the first conductive liquid medium in the cis region, has a net neutral charge or net positive charge. It will be appreciated that in some embodiments, the fusion reporter protein to be analyzed can be provided in the trans region and, upon application of the electrical potential, the flexible tail domain enters the nanopore from the trans side of the system. As indicated above, the blocking domain with a stably folded tertiary structure has a diameter that exceeds a dimension within the nanopore tunnel, thus preventing complete translocation of the linear fusion reporter protein molecule through the nanopore.

Nanopore systems also incorporate structural elements to measure and/or apply an electrical potential across the nanopore-bearing membrane or film. For example, the system can include a pair of drive electrodes that drive current through the nanopores. Typically, the negative pole is disposed in the cis region and the positive pole is disposed in the trans region. Additionally, the system can include one or more measurement electrodes that measure the current through the nanopore. These can include, for example, a patch-clamp amplifier or a data acquisition device. For example, nanopore systems can include an Axopatch-200B patch-clamp amplifier (Axon Instruments, Union City, Calif.) to apply voltage across the bilayer and measure the ionic current flowing through the nanopore. For example, in some embodiments, the applied electrical field includes a direct or constant current that is between about 10 mV and about 1 V. In some embodiments that include protein-based nanopores embedded in lipid membranes, the applied current includes a direct or constant current that is between about 10 mV and 300 mV, such as about 10 mV, 20 mV, 30 mV, 40 mV, 50 mV, 60 mV, 70 mV, 80 mV, 90 mV, 100 mV, 110 mV, 120 mV, 130 mV, 140 mV, 150 mV, 160 mV, 170 mV, 180 mV, 190 mV, 200 mV, 210 mV, 220 mV, 230 mV, 240 mV, 250 mV, 260 mV, 270 mV, 280 mV, 290 mV, 300 mV, or any voltage therein. In some embodiments, the applied electrical field is between about 40 mV and about 200 mV. In some embodiments, the applied electrical field includes a direct or constant current that is between about 100 mV and about 200 mV. In some embodiments, the applied electrical direct or constant current field is about 180 mV. In other embodiments where solid state nanopores are used, the applied direct or constant current electrical field can be in a similar range as described, up to as high as 1 V. As will be understood, the voltage range that can be used can depend on the type of nanopore system being used and the desired effect.

Persons of skill in the art will readily appreciate that the reverse electrical potential as the values and ranges described above can also be applied.

In some embodiments, the electrical potential is not constant, but rather is variable about a reference potential.

Methods

In another aspect, the disclosure provides methods of utilizing the described fusion reporter proteins in a nanopore system to determine a characteristic of the fusion reporter protein. This, in turn, can be extended to characterize and monitor activity in biological systems, such as cells, cell extracts, and other complex in vitro formulations incorporating biological reagents. As indicated above, the methods have the capacity to be scaled up and performed in a multi-flex format.

In one embodiment, the disclosure provides a method of characterizing biological activity of one or more cells in a nanopore system. A nanopore system referred to in this context comprises a nanopore disposed in a barrier defining a cis side and a trans side, wherein the cis side comprises a first conductive liquid medium and the trans side comprises a second conductive liquid medium, and wherein the nanopore comprises a tunnel that provides liquid communication between the cis side and the trans side. The method comprises:

providing a fusion reporter protein as described above into the first conductive liquid medium of the cis side of the nanopore system;

initiating translocation of the flexible tail domain of the fusion reporter protein through the nanopore tunnel, wherein the blocking domain of the fusion reporter protein has a diameter that exceeds the diameter of the nanopore tunnel at its narrowest point;

measuring an ion current between the first conductive liquid medium and the second conductive liquid medium when the flexible analyte domain of the fusion reporter protein is in the tunnel of the nanopore; and

detecting an ion current pattern associated with a structural characteristic of the flexible analyte domain of the fusion reporter protein.

As indicated above, the flexible tail domain is the first to interact with the nanopore tunnel, resulting in the flexible tail domain threading through the nanopore tunnel followed by the flexible analyte domain of the fusion reporter protein. Due to the diameter of the blocking domain, the blocking domain is pulled against the nanopore, e.g., the outer rim or vestibule, but maintains its tertiary structure and does not pass further into the nanopore. This pauses movement of the flexible domains within the nanopore, leaving the flexible analyte domain in a section of the nanopore tunnel where it can influence the detectable ion current, thereby providing a unique ion current pattern associated with it structural characteristics (e.g., its sequence or modification status).

The one or more cells can be a plurality of cells of the same type, e.g., multiple cells of the same lineage and cultured under the same conditions. Alternatively, the one or more cells can comprise different cells of distinct lineages (e.g., cells of different cell lines or cells from different source organisms), or the same or similar cells from the same lineage but to distinct experimental conditions.

In some embodiments, the fusion reporter protein is expressed in a cell from a nucleic acid comprising a first sequence that encodes the fusion reporter protein and a second sequence comprising a promoter sequence and/or an enhancer sequence operatively linked to the first sequence. Such embodiments can be useful to assay the activity of the promoter and/or enhancer sequence, i.e. the capacity to promote expression of the operatively linked encoding sequence, within the context of the target cell(s) under defined conditions. This has useful implications for determining the regulatory capacity of promoters in the presence of appropriate transcription factors within the target cellular environment(s). In some embodiments the method comprises expressing the fusion reporter protein in the one or more cells. The flexible analyte domain of the expressed fusion reporter protein can comprise a barcode amino acid sequence and the ion current pattern that is detected in the nanopore system can be associated with the structural characteristics of the barcode amino acid sequence. This allows for a correlation of the barcode amino acid sequence with aspects of the experimental design, for example, the activity of the particular promoter sequence within the target cell and/or experimental conditions imposed during expression. Detection of the ion current pattern indicates that the associated promoter and/or enhancer sequence operatively linked to the sequence encoding the fusion reporter protein with the barcode sequence is biologically active in the cell.

Furthermore, in some embodiments analysis can extend beyond detection of activity versus no activity (i.e., expression versus no expression). Instead, the further method encompasses determining the expression level of the fusion reporter protein in one or more cells. Such quantification can be performed by determining the average time between successive captures of the barcode sequence within the nanopore under predetermined conditions. In another embodiment, the overall number of detection events of one or more unique barcodes can be determined per nanopore over a period of time under predetermined conditions. With higher expression levels of the fusion reporter protein from the operatively linked promoter and/or enhancer sequence, the quantity of fusion reporter proteins in the nanopore-based assay is increased. A higher quantity of fusion reporter proteins results in an increased rate of fusion reporter protein capture by the nanopore, and hence increased rate of observation of the identifying ion current pattern that is associated with the barcode sequence. These measures of fusion reporter protein capture by nanopores can then be compared to a standard control or curve that establishes such measures of capture under similar or the same nanopore system operating conditions.

In other embodiments, the flexible analyte domain comprises a target sequence for post-translation modification. The structural characteristic associated with the detective ion current pattern observed in a nanopore system can be the presence or absence of a modification at the target sequence in the flexible analyte domain. In such embodiments, the activity of the biological system(s) encompassed by the target one or more cells can be assayed for the capacity to modify the target sequence of the translated fusion reporter protein. For example, this approach can be used to determine the presence of protein-modifying enzymes, such as kinases, phosphorylases, methylases, and the like, within one or more defined cellular contexts. This disclosure encompasses target sequences for any post-translation modification known in the art. Exemplary, nonlimiting post-translation modifications include phosphorylation, methylation, glycosylation, acetylation, lipidation, nitrosylation, and the like. Target sequences for such modifications including target sequences specifically recognized by known enzymes are familiar to persons of ordinary skill in the art and are encompassed by the present disclosure. In further embodiments this approach can be used to quantify the activity or capacity of the one or more cells to implement the post-translation modification. This can be accomplished by quantifying the degree of post-translation modification in a batch of fusion reporter proteins with the same target sequence. Accordingly, instead of detecting the presence or absence of post-translation modifications, the method is applied to characterize the relative activity of the agents that impose the post translation modification. As indicated above, the degree of modification can be quantified by detecting the relative frequency of detection events or the average time between successive captures by the nanopore. The results can be compared to standard curves or comparison controls to ascertain the relative modification activity of the cellular environment.

As indicated above, the disclosed methods can be scaled up and even multiplexed for broader analysis of biological systems within the same nanopore-assay. For example, a plurality of distinct fusion reporter proteins that comprise flexible analyte domains with different amino acid sequences can be employed. The different amino acid sequences can represent different barcodes (i.e., the flexible analyte domain can contain a degenerate sequence), where each barcode is associated with a different experimental condition. Such experimental conditions can be different promoter sequences driving expression of the fusion reporter protein, different target cells expressing a fusion reporter protein, different culture environments (e.g., drug treatments conditions) of the cells expressing the fusion reporter proteins, and the like. The flexible analyte domain has the capacity to contain extensive barcode variability, where each individual barcode can be uniquely identified and/or quantified, and associated with a unique experimental condition for comparison.

In another embodiment encompassed by the disclosure, the different fusion reporter proteins have flexible analyte domains with different target sequences for post-translation modifications. The panel of different fusion reporter proteins can represent a survey of a cell's (or multiple cells') capacity to impose post-translation modifications. In some embodiments, the plurality of distinct fusion reporter proteins with analyte domains having different amino acid sequences are expressed in different cells or cell-types. This allows simultaneous characterization and comparison of multiple cell-types in a single assay.

While the above methods are generally described in the context of assessing biological systems of a cell or a plurality of cells, a person of ordinary skill in the art will readily appreciate that the described methods can be modified to address acellular biological systems. For example using cell lysates or in vitro-assembled reaction systems, encoding the fusion reporter proteins can be transcribed and translated. In other embodiments, fusion reporter proteins previously translated in a cell for in vitro can be exposed to an environment that may or may not contain agents that can modified proteins at a target site. For example, fusion reporter proteins with flexible analyte domains containing modification target sequences can be exposed to different reaction conditions and/or different putative modifying enzymes. The reaction conditions and/or different modifying enzymes can be assayed for activity on the target sites included in the flexible analyte domains. Accordingly, the present disclosure encompasses methods characterize and monitor biological activity in one or more acellular biological environments using a nanopore system.

### General Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed in the specification, examples, and appended claims are provided here. The definitions are provided to aid in describing particular embodiments and are not intended to limit the claimed invention, as the scope of the invention is limited only by the claims.

The use of the term “or” in the claims and specification is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an open and inclusive sense as opposed to a closed, exclusive or exhaustive sense. For example, the term “comprising” can be read to indicate “including, but not limited to.” The term “consists essentially of” or grammatical variants thereof indicate that the recited subject matter can include additional elements not recited in the claim, but which do not materially affect the basic and novel characteristics of the claimed subject matter.

Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being typical. The term polypeptide or protein as used herein encompasses any amino acid sequence and includes modified sequences such as glycoproteins. The term polypeptide, unless noted otherwise, is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine (S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

Reference to sequence identity addresses the degree of similarity of two polymeric sequences, such as protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following describes the design and implementation of exemplary protein reporter constructs, referred to as Nanopore-addressable protein Tags Engineered as Reporters (nanoporeTERs or NTERs) provided by the present disclosure. The disclosed NTER design can be used with any available nanopore sensor and can be multiplexed for direct protein reporter detection without the need for other specialized equipment or laborious sample preparation prior to analysis.

In the first implementation of the design, a set of NanoporeTER proteins was engineered that could be expressed in E. coli and easily detected by nanopore sensors. The initial NTER design was based on the synthetic protein construct ‘S1’, which was previously developed for unfoldase-mediated nanopore analysis (see, e.g., Nivala, J., et al., “Unfoldase-mediated protein translocation through an α-hemolysin nanopore,” Nat. Biotechnol. 31, 247-250 (2013). doi:10.1038/nbt.2503; and Nivala, J., et al., “Discrimination among protein variants using an unfoldase-coupled nanopore,” ACS Nano 8, 12365-12375 (2014), each of which is incorporated herein by reference in its entirety). Si contains a small, folded domain (Smt3) along with a flexible, negatively-charged 65 amino acid C-terminal ‘tail’ composed of glycine, serine, and acidic amino acid residues, in addition to an 11 amino acid ssrA tag (Baker, T. A. & Sauer, R. T., “ClpXP, an ATP-powered unfolding and protein-degradation machine,” Biochim. Biophys. Acta—Mol. Cell Res. 1823, 15-28 (2012) incorporated herein by reference in its entirety). The tail's lack of structure and net negative charge promotes capture of the protein in a nanopore sensor under an applied voltage. The ssrA tag allows for ClpX-mediated unfolding and translocation of the Smt3 domain, which otherwise inhibits translocation of S1 through the nanopore. For use as a reporter protein in E. coli, the S1 protein was modified in two ways (FIG. 1A and Table 1). First, the ssrA tag was replaced with additional glycine/serine/acidic residues to preserve its nanopore threading activity but preventing targeting of the protein for degradation by ClpXP in vivo. Second, an N-terminal OsmY domain (see, e.g., Yim, H. H. & Villarejo, M., “osmY, a new hyperosmotically inducible gene, encodes a periplasmic protein in Escherichia coli,” J. Bacteriol. 174(11), 3637-3644 (1992)) was added. In E. coli, OsmY-tagged proteins are secreted into the extracellular medium. This design is based on a hypothesis that that secretion would facilitate NTER nanopore analysis by avoiding the need to lyse cells, thereby simultaneously reducing both experimental labor and signal noise that could be generated by non-specific interaction of intracellular molecular species (e.g. DNA, RNA, and other proteins) with the nanopores during analysis. Experiments in BL21 (DE3) E. coli showed that expression of this modified version of S1, which is referred to here as ‘NTER00’, resulted in secretion of the protein into the medium, as detected by SDS-PAGE analysis (FIG. 4A).

TABLE 1
sequence design of nanoporeTER constructs.

| | | SEQ ID |
|---|---|---|
| Name | Sequence | NO: |
| General sequence | MTMTRLKISKTLLAVMLTSAVATGSAYAENNAQTTNESAGQ | 1 |
| of NanoporeTER | KVDSSMNKVGNFMDDSAITAKVKAALVDHDNIKSTDISVKT | |
| construct design* | DQKVVTLSGFVESQAQAEEAVKVAKGVEGVTSVSDKLHVR | |
| | DAKEGSVKGYAGDTATTSEIKAKLLADDIVPSRHVKVETTD | |
| | GVVQLSGTVDSQAQSDRAESIAKAVDGVKSVKNDLKTK | |
| | MGHHHHHHHHHHGS \| | |
| | LQDSEVNQEAKPEVKPEVKPETHINLKVSDGSSEIFFKIKKTT | |
| | PLRRLMEAFAKRQGKEMDSLRFLYDGIRIQADQAPEDLDME | |
| | DNDIIEAHREQI \| | |
| | XXXXXXXXXXXXXXXXXXXXXXXXXXXXXXX \| | |
| | DGGSSGGSGGDGSSGDGGSDGDSDGSDGDGDSDGDDGGDD | |
| | EDDGSDD | |

Barcode sequence of analyte domain**

| | | |
|---|---|---|
| NTER00 | GGGGSSGGSGGSGSSGDGGSSGGSGGSGSSG | 2 |

Barcode sequences of YYY mapping mutants

| | | |
|---|---|---|
| NTER01 | YYYGSSGGSGGSGSSGDGGSSGGSGGSGSSG | 3 |
| NTER02 | GGYYYSGGSGGSGSSGDGGSSGGSGGSGSSG | 4 |
| NTER03 | GGGGYYYGSGGSGSSGDGGSSGGSGGSGSSG | 5 |
| NTER04 | GGGGSSYYYGGSGSSGDGGSSGGSGGSGSSG | 6 |
| NTER05 | GGGGSSGGYYYSGSSGDGGSSGGSGGSGSSG | 7 |
| NTER06 | GGGGSSGGSGYYYSSGDGGSSGGSGGSGSSG | 8 |
| NTER07 | GGGGSSGGSGGSYYYGDGGSSGGSGGSGSSG | 9 |
| NTER08 | GGGGSSGGSGGSGSYYDGGSSGGSGGSGSSG | 10 |
| NTER09 | GGGGSSGGSGGSGSSGDYYSSGGSGGSGSSG | 11 |
| NTER10 | GGGGSSGGSGGSGSSGDGYYYGGSGGSGSSG | 12 |
| NTER11 | GGGGSSGGSGGSGSSGDGGSYYYSGGSGSSG | 13 |
| NTER12 | GGGGSSGGSGGSGSSGDGGSSGYYYGSGSSG | 14 |
| NTER13 | GGGGSSGGSGGSGSSGDGGSSGGSYYYGSSG | 15 |
| NTER14 | GGGGSSGGSGGSGSSGDGGSSGGSGGYYYSG | 16 |
| NTER15 | GGGGSSGGSGGSGSSGDGGSSGGSGGSGYYY | 17 |

Barcode sequences of homopolymer mutants

| | | |
|---|---|---|
| NTER A | GGAAAAAAAAASGSSGDGGSSGGSGGSGSSG | 18 |
| NTER D | GGDDDDDDDDDSGSSGDGGSSGGSGGSGSSG | 19 |
| NTER E | GGEEEEEEEEESGSSGDGGSSGGSGGSGSSG | 20 |
| NTER G | GGGGGGGGGGGSGSSGDGGSSGGSGGSGSSG | 21 |
| NTER H | GGHHHHHHHHHSGSSGDGGSSGGSGGSGSSG | 22 |
| NTER M | GGMMMMMMMMMSGSSGDGGSSGGSGGSGSSG | 23 |
| NTER N | GGNNNNNNNNNSGSSGDGGSSGGSGGSGSSG | 24 |
| NTER P | GGPPPPPPPPPSGSSGDGGSSGGSGGSGSSG | 25 |
| NTER Q | GGQQQQQQQQQSGSSGDGGSSGGSGGSGSSG | 26 |
| NTER R | GGRRRRRRRRRSGSSGDGGSSGGSGGSGSSG | 27 |
| NTER S | GGSSSSSSSSSSGSSGDGGSSGGSGGSGSSG | 28 |
| NTER T | GGTTTTTTTTTSGSSGDGGSSGGSGGSGSSG | 29 |

Barcode sequences of PKA motif mutants

| | | |
|---|---|---|
| NTER PKA | GGRRGSYYSGGSGSSGDGGSSGGSGGSGSSG | 30 |
| NTER PKA | GGRRGEYYSGGSGSSGDGGSSGGSGGSGSSG | 31 |
| phosphomimetic | | |

*In the sequence of the NanoporeTER, the domains are separated by a vertical line “|”, These domains, in order, are: OsmY domain (SEQ ID NO: 32)|His-tag (SEQ ID NO: 33)|Smt3 domain (SEQ ID NO: 34)|Analyte domain (indicated with Xs to indicate a variable region, generally containing a barcode or enzymatic targeting sequence addressed in the remainder of the table)|PolyGSD tail domain (SEQ ID NO: 35).
**Only the sequences of the analyte domains, i.e. the variable region in the top sequence with a series of X residues, are shown. NTER contains in the indicated analyte domain sequence integrated into the constructs sequence listed at the top.

Next, the secreted NTER00 was purified by immobilized metal affinity chromatography (IMAC) and then assessed for whether the NTER could be detected on a MinION® nanopore platform. To do this, an unmodified R9.4.1 flow cell (which uses a variant of the CsgG pore protein; see, e.g., Goyal, P. et al. “Structural and mechanistic insights into the bacterial amyloid secretion channel CsgG,” Nature 516, 250-253 (2014)) was used and a custom MinION® run script (see Example 1—Methods). The script applies a constant voltage of −180 mV to all the active pores on the flow cell and statically flips the voltage in the reverse direction in 15 second cycles (i.e. 10 seconds ‘ON’ at −180 mV and 5 seconds ‘OFF’ or in ‘Reverse’, see FIG. 1E). The typical R9.4.1 open pore current level at −180 mV and 500 mM KCl is ˜220 pA. As expected, in these conditions and following the introduction of NTER00 into the flow cell at a concentration of 0.5 uM, the current level during each −180 mV portion of the voltage cycle typically underwent a stepwise drop from the open pore value to a consistent lower ionic current state (see, e.g., FIG. 1E), signaling a putative capture of an NTER within the pore. This current drop was reversible (back to open pore) following reversal of the voltage. It was also found that the average time of the open pore prior to transitioning to the lower ionic current state was NTER concentration dependent (FIG. 1F). These observations are consistent with a model in which the negatively-charged NTER polyGSD tail is electrophoretically captured in the pore under the applied voltage (−180 mV), and can be ejected from the pore by reversal of the electric field.

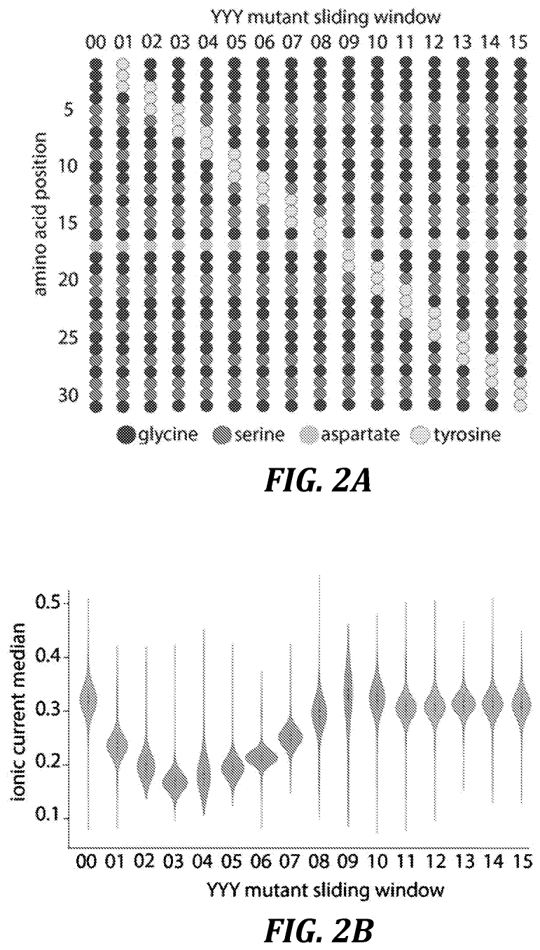

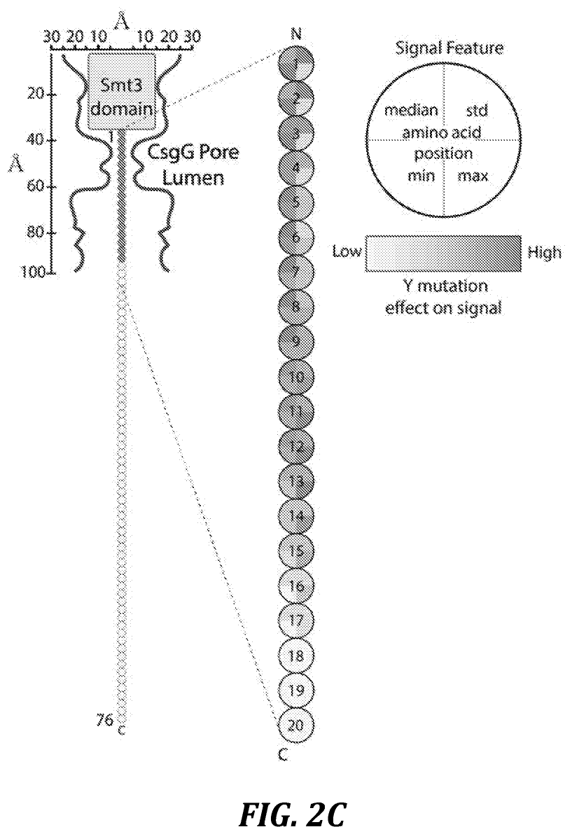

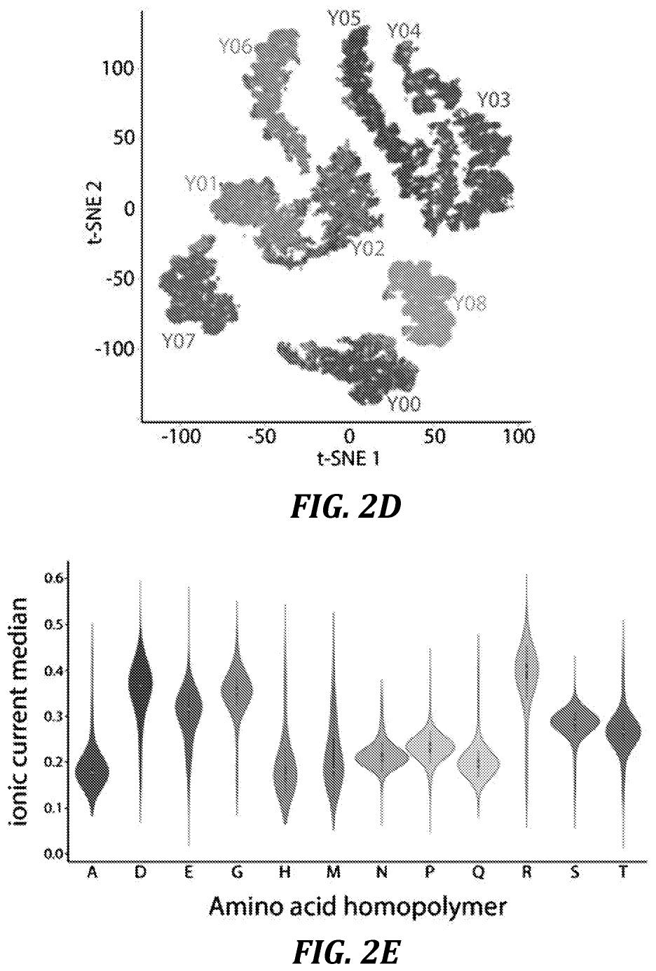

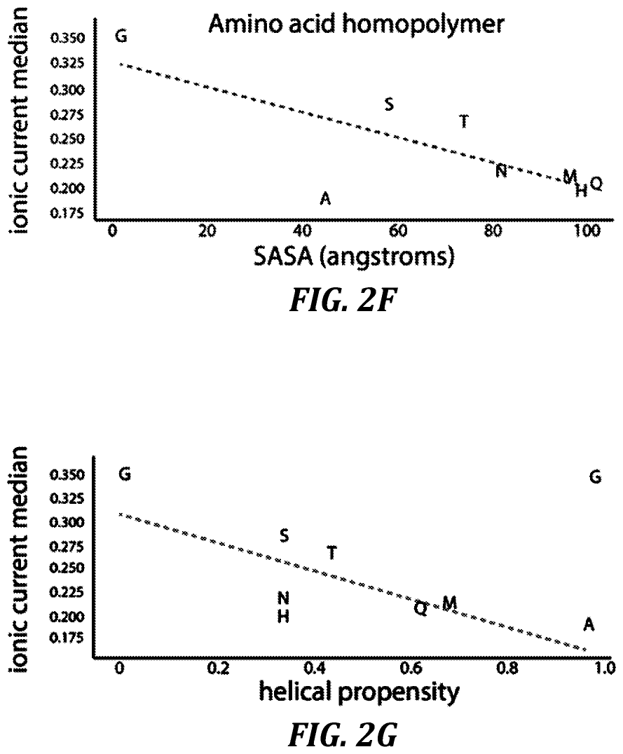

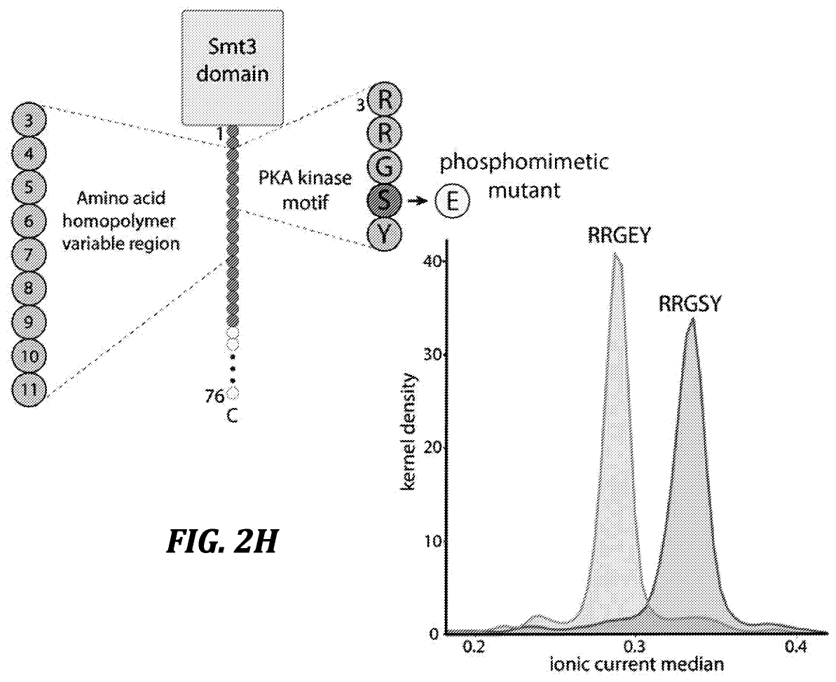

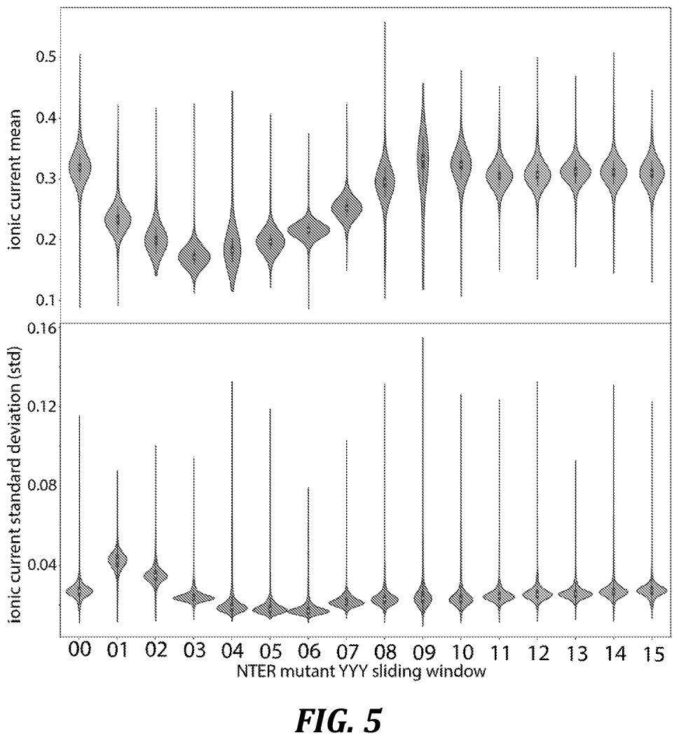

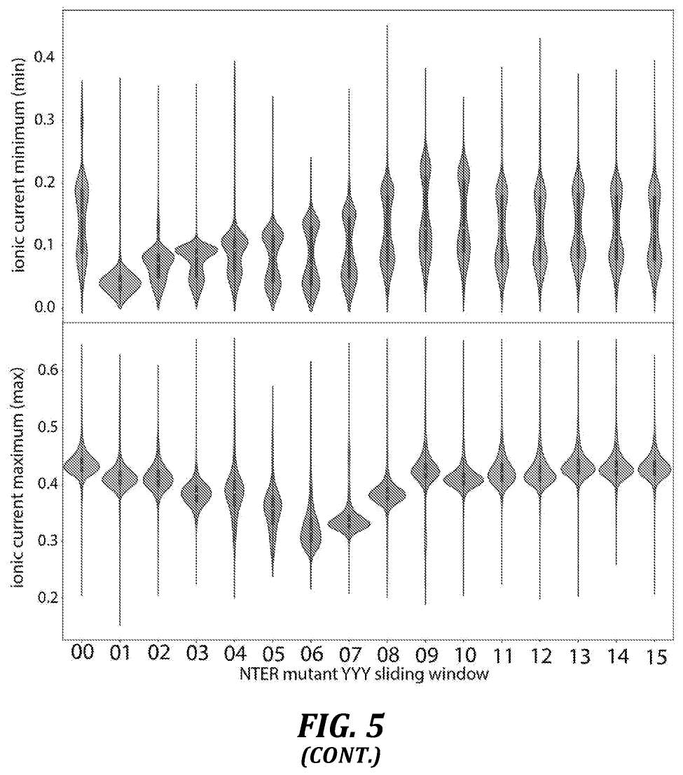

In view of this model, it was postulated that the ionic current characteristics of the NTER00 capture state should be dependent upon the amino acid sequence of the residues residing within the pore's sensitive limiting constriction. To test this, a series of NTER mutants (NTER01-15) was constructed in which a sliding three residue region of the polyGSD sequence was mutated to tyrosines (FIG. 2A and Table 1). Tyrosines were chosen because their larger side chain structure was predicted to decrease the ionic current flow through the pore relative to the glycines and serines of NTER00 when captured within the pore. Following purification and MinION® analysis of NTERs 01-15, the capture state was found to be NTER mutant-dependent up to NTER08, after which NTER mutants 09-15 were observed to have signal characteristics indistinguishable from NTER00 (FIGS. 2B, 2C, 2D, and 5). These results support a model in which the first ˜17 amino acids of the polyGSD tail reside with the CsgG nanopore's sensitive region and contribute to its ionic current signature during a capture event. It also sets an upper bound to the number of possible NTER barcodes around 20¹⁷ to ˜10²².

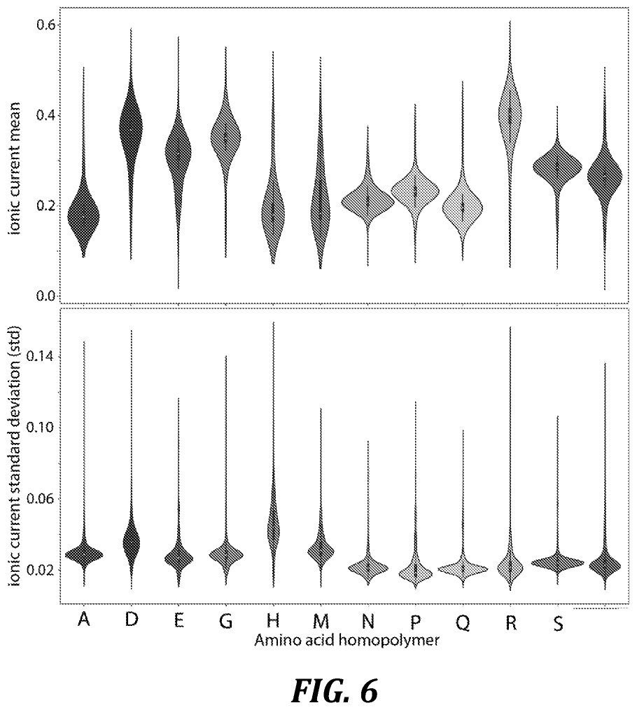

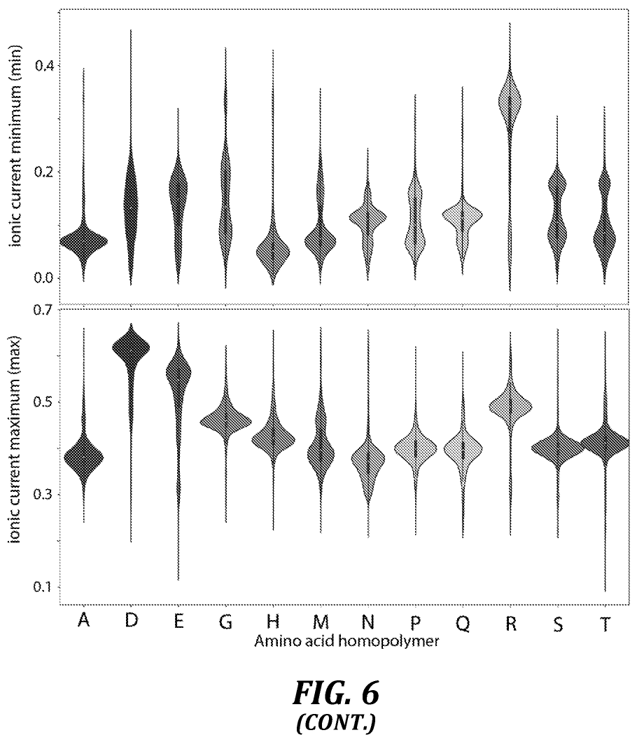

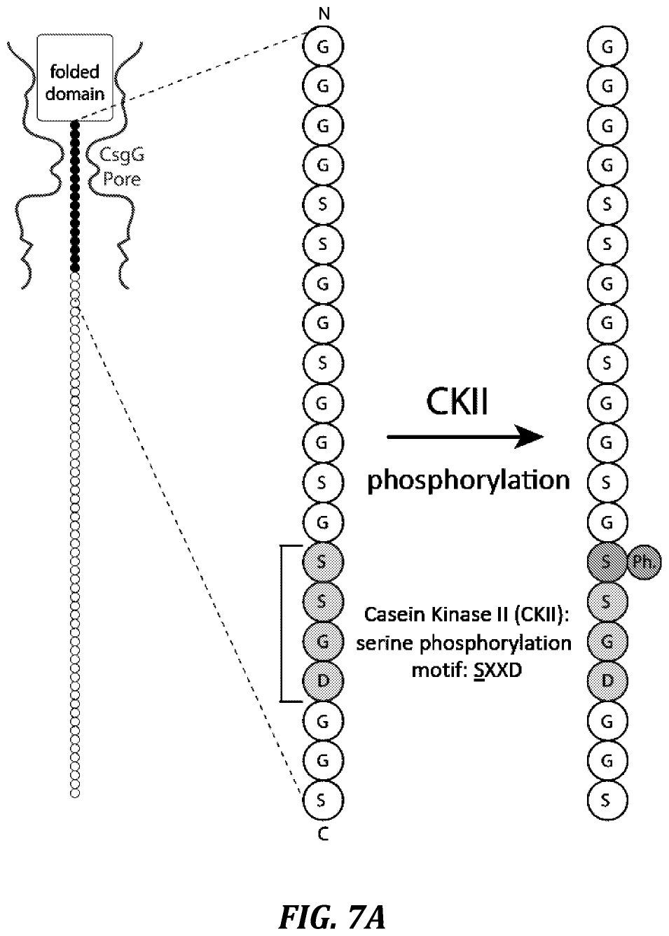

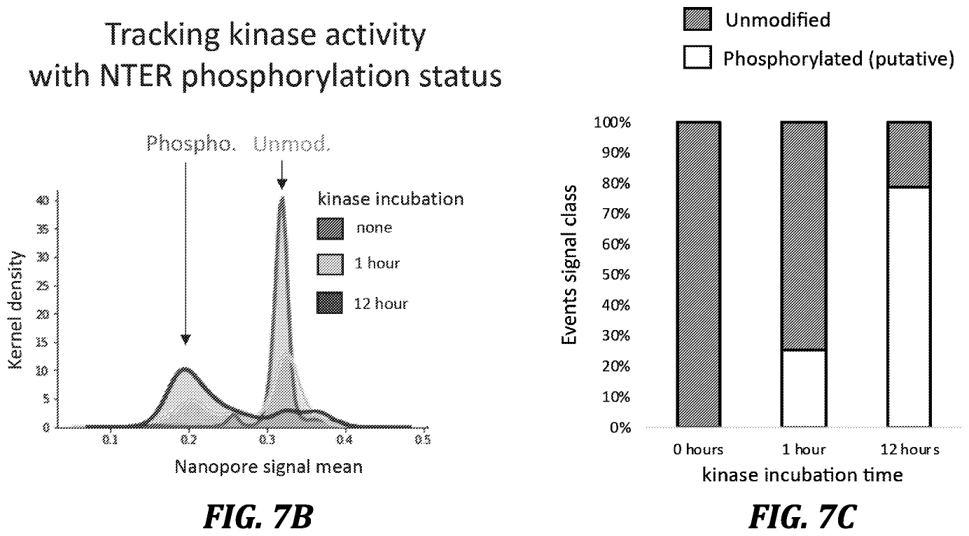

After determining the number of amino acids that contribute to the NTER nanopore signal (the NTER sequence space), the next step was to determine how different amino acid types modulate the ionic current through the pore. These results help define the possible future NTER signal space. To investigate this, NTER variants were constructed in which positions 3-12 within the polyGSD region were mutated to all the 20 possible standard amino acid homopolymers (see TABLE 1). FIGS. 2E and 6 show the signal features of the ionic current levels for 12 out of the 20 NTER homopolymer mutants (the homopolymers C, F, I, K, L, V, W, and Y, most of which have significant hydrophobic character, did not express sufficient soluble protein). To see how the different amino acid physical properties contribute to the NTER ionic current, certain specific properties were investigated to determine whether they correlate with different signal features. While no strong correlations were found across all the 15 amino acid types, a strong correlation was observed between the mean current level with both the amino acid volume and the amino acid helical propensity within the uncharged amino acid types (R correlation=˜0.75, FIGS. 2F and 2G).

Next, to probe the potential of this method to resolve between amino acid barcodes with subtler sequence differences (for example, point mutations or post-translational modifications), two additional NTER barcodes based on the protein kinase A (PKA) phosphorylation motif (see, e.g., Taylor, S. S., et al., “PKA: A portrait of protein kinase dynamics,” Biochimica et Biophysica Acta—Proteins and Proteomics 1697 (1-2), 259-269 (2004), incorporated herein by reference in its entirety) were cloned and tested. The first PKA-based barcode contained a canonical PKA motif (RRGSY), while the second had a single amino acid difference (RRGEY) that mimics the PKA motif s phosphorylated serine state in structure and charge (commonly referred to as a ‘phosphomimetic’, see TABLE 1 and FIG. 2H). Following purification and MinION® analysis of these two NTERs, the phosphomimetic barcode was found to be distinguishable from the canonical PKA motif barcode, as the two barcodes typically had substantially different nanopore ionic current state medians (FIG. 2H). These results demonstrate that NanoporeTERs can be used to assess the activity of enzymes that regulate specific post-translational modifications, such as phosphorylation and methylation.

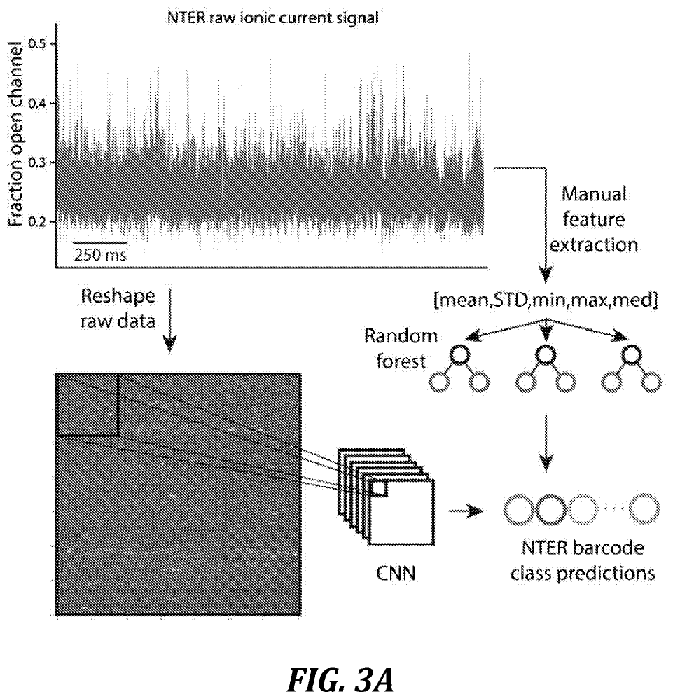

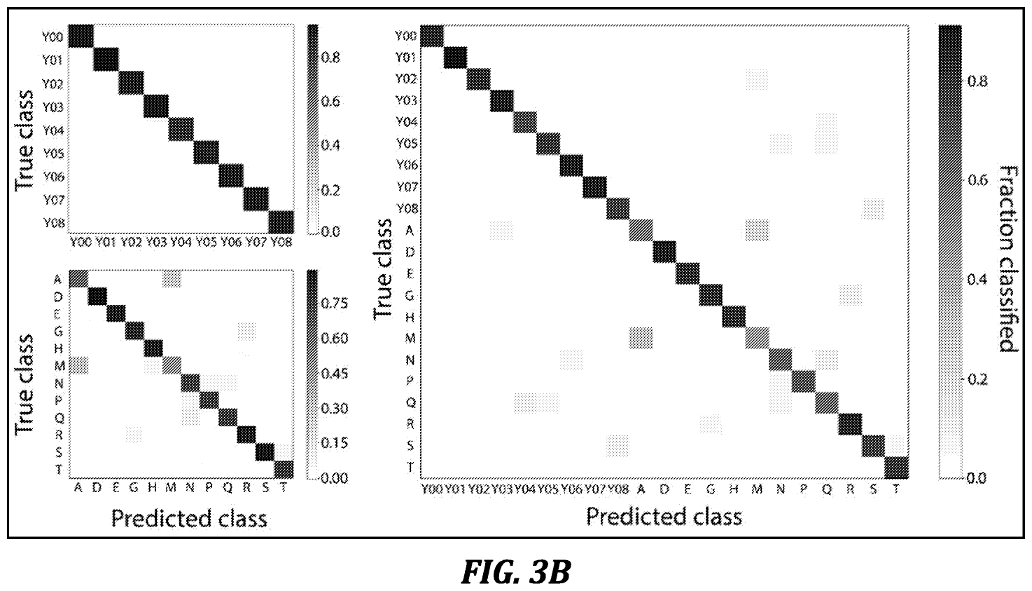

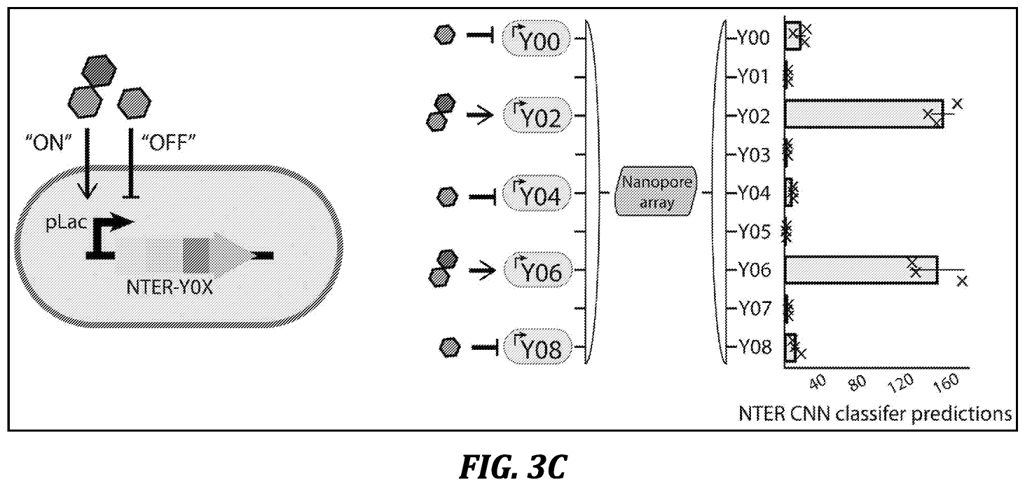

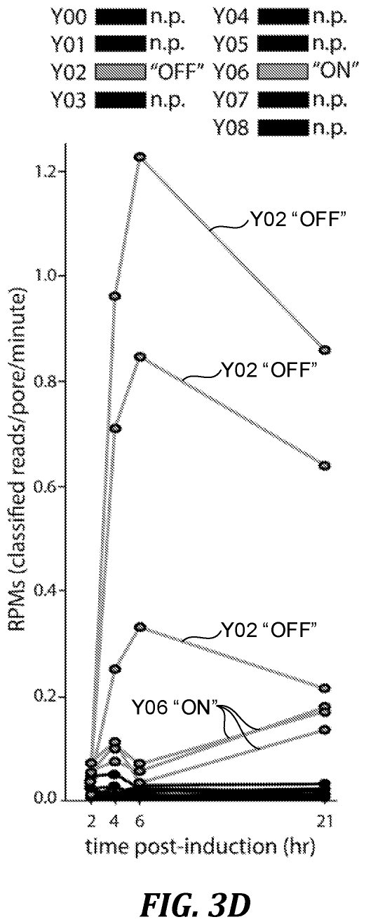

Finally, having explored the potential NTER barcode sequence space, signal space, and sensitivity to single residue modifications, proof-of-principle NTER applications was demonstrated for multiplexed tracking of gene expression. To accomplish this, supervised machine learning was first used to train classifiers that could accurately discriminate amongst combinations of the NTER barcodes explored above. Using either a set of engineered signal features as input to a Random Forest (RF) classifier or the raw ionic current signal directly into a Convolutional Neural Network (CNN) (FIG. 3A), purified NTER datasets described above were used for model training and validation. Both models achieved similar accuracies that ranged from ˜80-90% depending on the model hyperparameters and barcode set (FIG. 3B; see also EXAMPLE 1—Methods).

The best performing CNN that was trained on NTER Nos. Y00-08 was used to determine the relative NTER expression levels within bacterial cultures composed of mixed populations of strains engineered with different NTER-tagged plasmid-based circuits. To do this, independent mono-barcoded cultures were grown overnight with NTER expression either induced or inhibited (by the addition of IPTG or glucose, respectively). In the morning, just prior to nanopore readout, the cultures were mixed into a single solution and diluted into MinION® running buffer and loaded directly into a flow cell for analysis. Importantly, the results showed higher classification counts for the NTER barcodes for which expression was induced (NTER Nos. 02 and 06), and lower levels for strains that were inhibited (glucose: NTER Nos. 00, 04 and 08) or not present at all in the mixed population (NTER Nos. 01, 03, 05, and 07) for all replicates (FIG. 3C). We then conducted a time course experiment in which we tracked expression of two different NTERs over multiple hours, one of which was induced with IPTG (NTER06), and the other which NTER expression was inhibited with glucose (NTER02). Again, cultures were grown independently, but then mixed just prior to nanopore readout. FIG. 3D shows the results of this time course (and replicates) following MinION® analysis at 2, 4, 6, and 21 hour timepoints following induction (NTER06) or inhibition (NTER02) of the NanoporeTER circuit. Again, the rate of NTER classification was higher for the induced NTER06 circuits, compared to the uninduced NTER02 circuits. Importantly, leaky expression of NTER02 was still detectable over the background false-positive classification rates for the NTER barcodes that were not present at all in the experiment (00, 01, 03, 04, 05, 07 and 08). These results demonstrate that NanoporeTERs can be used as reliable reporters of relative protein expression levels.

In conclusion, this work demonstrates the design and implementation of a new class of multiplexable protein reporters (NanoporeTERs or NTERs) that can be analyzed using commercially available nanopore sensors, e.g., the Oxford Nanopore Technologies (ONT) MinION®. While this work addresses a set ˜20 orthogonal NanoporeTERs, this number can be increased significantly with the following strategies: 1) high-throughput methods to empirically characterize more barcode sequences for classifier training, 2) engineering NanoporeTERs to contain multiple barcode regions that can be consecutively readout with the aid of processive motor proteins (see, e.g., Nivala, J., et al., “Unfoldase-mediated protein translocation through an α-hemolysin nanopore,” Nat. Biotechnol. 31, 247-250 (2013). doi:10.1038/nbt.2503; and Nivala, J., et al., “Discrimination among protein variants using an unfoldase-coupled nanopore,” ACS Nano 8, 12365-12375 (2014), each of which is incorporated herein by reference in its entirety) or voltage-mediated translocation (Rodriguez-Larrea, D. & Bayley, H., “Multistep protein unfolding during nanopore translocation,” Nat. Nanotechnol. 8, pages 288-295 (2013), incorporated herein by reference in its entirety), which would allow the number of orthogonal NTERs to scale exponentially with the number of individually characterized barcodes, and 3) semi-supervised machine learning models trained to accurately predict the sequence of empirically uncharacterized NTER barcodes given only their nanopore signal (Sutskever, I., et al., “Sequence to Sequence Learning with Neural Networks,” In Advances in neural information processing systems, 3104-3112 (2014), incorporated herein by reference in its entirety). Considering their modular design, NanoporeTER can be used in any cell expression system of choice. The choice of cell expression system will impact the design of the NanoporeTER only insofar as the choice of an appropriate secretion domain, if a secretion domain is desired to facilitate easy isolation of the NanoporeTER reporter constructs for subsequent nanopore-based analysis. Many such N-terminal secretion domains have been characterized in a range of diverse organisms. See, e.g., Olczak, M. & Olczak, T. “Comparison of different signal peptides for protein secretion in nonlytic insect cell system,” Anal. Biochem. 359(1), 45-53 (2006); Bitter, G. A., et al., “Secretion of foreign proteins from Saccharomyces cerevisiae directed by alpha-factor gene fusions,” Proc. Natl. Acad. Sci. 81(17), 5330-5334 (1984); and, Attallah, C., et al., “A highly efficient modified human serum albumin signal peptide to secrete proteins in cells derived from different mammalian species,” Protein Expr. Purif. 132, 27-33 (2017); each of which is hereby incorporated by reference in its entirety.

NanoporeTER reporter constructs can be employed for many applications, including simultaneously reading the protein-level outputs of many genetically engineered circuit components in one-pot, enabling more efficient debugging and tuning than current analysis methods. For instance, in comparison to traditional sets of fluorescent protein reporters, NanoporeTERs have a (potentially much) larger sequence and signal space that allows for the simultaneous analysis of a greater number of unique genetic elements in a single experiment (multiplexing). While RNA-seq is an alternative strategy that can be used to measure the transcriptional output of many circuits in parallel with high-throughput DNA sequencing technology, methods incorporating the NanoporeTER reporter designs have the advantages of 1) little to no sample preparation, which makes it more amenable to automation and reduces both time to analysis (latency) and cost, and 2) direct detection of outputs at the protein level. The latter advantage provides new opportunities to custom engineer reporters with NTER barcodes that can report on both protein expression and specific post-translational modifications simultaneously. This capability is especially useful as the nascent field of synthetic protein-level circuit engineering advances.

### EXAMPLES

The following example is provided for the purpose of illustrating, not limiting, the disclosure.

### Example 1

### Methods and Materials

NanoporeTER Construction, Cloning, Expression, and Purification

The initial NanoporeTER protein was constructed with a gBlock (Integrated DNA Technologies) composed of the Smt3 and tail sequence and cloned into plasmid pCDB180 downstream of the OsmY domain. The Q5 site-directed mutagenesis method (New England Biolabs) was used to generate the different NTER barcode mutants. All cloning was performed using the 5-alpha competent E. coli strain following NEB's cloning protocol (New England Biolabs). Sequence verification was obtained through Genewiz Inc. Expression of the NanoporeTER protein was done in BL21 (DE3) E. coli strain using Overnight Express instant TB medium (Novagen).

Proteins were purified via immobilized metal affinity chromatography (IMAC) using TALON metal affinity cobalt resin (Takara). The purification used the associated buffer set from Takara, following their specified protocol. Proteins were concentrated using Amicon Ultra 0.5 mL centrifugal filters with Ultracel 30K (Amicon). The final concentration of proteins averaged ˜7 mg/ml from 5 mL overnight cultures. The purified proteins were stored for long-term storage at −80 C in 10 uL aliquots, as well as for short-term storage at 4 C.

Raw Culture Mixing Experiments

Cultures were picked from single colonies on plates and used to inoculate 3 mL LB supplemented with 0.5 mM IPTG and kanamycin (induced), or 3 mL LB supplemented with 0.2% glucose and kanamycin (uninduced). After overnight incubation at 37 C with shaking, cultures were equally mixed together in a total volume of 45 uL, 50 uL 4×C17 buffer, and 105 uL water (total volume 200 uL). This solution was then immediately loaded into a MinION® flow cell for analysis.

Time Course

Time course experiments were performed by diluting 30 uL of overnight cultures (LB) into 3 mL fresh LB supplemented with 0.5 mM IPTG and kanamycin (induced), or 3 mL fresh LB supplemented with 0.2% glucose and kanamycin (uninduced). The cultures were placed in a shaker/incubator at 37 C to allow for culture growth. Time-points were then collected at 2, 4, 6, and 21-hour. At each time point, cultures were equally mixed together in a total volume of 10 uL, 50 uL 4×C17 buffer, and 140 uL water (total volume 200 uL). This solution was then immediately loaded into a MinION® flow cell for analysis.

MinION® Experiments

All experiments were performed with unmodified R9.4.1 MinION® flow cells (Oxford Nanopore Technologies (ONT)) by diluting analyte solution into C17 buffer for a final concentration of 0.5M KCl and 25 mM HEPES (pH 8), into the flow cell priming port. Flow cells were run on the MinION® at a temperature of 30° C. and a run voltage of −180 mV with a 10 khz sampling frequency and 15 second static flip frequency. Use of a modifiable MinKNOW® script (available from ONT) enabled voltage flipping cycle parameters to be set as well as collection of raw current data across the entire run. Individual flow cells could be reused for different analytes after flushing them with 1 mL C17 buffer three times between experiments. Flow cells were stored at 4° C. in C18 buffer (150 mM potassium ferrocyanide, 150 mM potassium ferricyanide, 25 mM potassium phosphate, pH8) when not in use.

Nanopore Signal Analysis, Quantification, and Classification

The analysis pipeline for a NanoporeTER sequencing run begins with extracting the segments of the raw nanopore signal that contain capture events. A capture is defined as a region where the signal current falls below 70% of the open pore current for a duration of at least one millisecond. The fractional current values (as compared to open pore current) computed from the segmentation process, as well as the start and end times of each capture, are saved in separate data files. This information is then passed through a general filter that separates putative NanoporeTER captures from noise captures based on features of the raw current (mean, median, min, max, standard deviation) as well as the duration of the capture. Captures that pass this initial filter are then fed into a classifier (Random Forest or Convolutional Neural Network (CNN)) and classified as a specific NTER barcode. The metadata for the captures within each NTER class are subsequently fed to a quantifier which calculates the average time elapsed between those captures and converts this time to the predicted NTER concentration using a standard curve.

Machine Learning Classifiers

Two different classifiers for NTER barcode discrimination were explored. The first, a Random Forest model, was implemented in scikit-learn (sklearn.ensemble.RandomForestClassifier). The second classifier was a CNN implemented in PyTorch. An 80/20 train/test split was used to generate the classification accuracy estimates and confusion matrix results. For both models, only the first two seconds of each capture were considered for analysis. The Random Forest was trained on an array composed of the mean, standard deviation, minimum, maximum, and median of that two second window. Default Random Forest hyperparameters were modified to: n_estimators=300 and max_depth=100. The CNN used the two seconds of raw signal directly as input following reshaping of the 1D signal into a 2D structure. The neural network was composed of four 2D convolutional layers each with ReLU activation and max pooling. These were followed by a fully connected layer which had a log-sigmoid activation function, and then a final output layer of the same size as the number of NTER classes considered in the experiment. Full model details and code can be found at github.com/uwmisl/NanoporeTERs.

While illustrative embodiments have been illustrated and described, it will be appreciated that various changes can be made therein without departing from the spirit and scope of the invention.

1. A fusion reporter protein comprising, in order:
a blocking domain with a stably folded tertiary structure;
a flexible analyte domain; and
a flexible tail domain, wherein the flexible tail domain has a net negative charge.

2. The fusion reporter protein of claim 1, wherein the flexible tail domain is configured to initiate translocation of the fusion reporting protein through a nanopore tunnel, and wherein the blocking domain is configured to have a diameter exceeding a diameter of the nanopore tunnel thereby preventing further translocation of the reporter protein through the nanopore tunnel when the blocking domain comes into contact with the nanopore.

3. The fusion reporter protein of claim 1, wherein the folded tertiary structure of the blocking domain has a diameter greater than about 1.5 nm.

4. The fusion reporter protein of claim 1, wherein the blocking domain has an amino acid sequence of between about 50 amino acids and about 250 amino acids.

5. The fusion reporter protein of claim 1, wherein the blocking domain comprises a small ubiquitin related modifier (SUMO)-like protein or a titan protein domain.

6. The fusion reporter protein of claim 5, wherein the SUMO-like protein domain is an Smt3 domain.

7.-9. (canceled)

10. The fusion reporter protein of claim 1, wherein the flexible analyte domain has an amino acid sequence of between about 15 and about 25 amino acids.

11. The fusion reporter protein of claim 1, wherein the flexible analyte domain has an amino acid sequence containing a uniquely identifiable barcode.

12. The fusion reporter protein of claim 1, wherein the flexible analyte domain has an amino acid sequence containing a target sequence for a post-translation modification.

13.-14. (canceled)

15. The fusion reporter protein of claim 1, wherein the flexible tail domain has an amino acid sequence with at least about 20 amino acids.

16. The fusion reporter protein of claim 1, wherein the flexible tail domain comprises a plurality of amino acids selected from glycine, serine, aspartic acidic, glutamic acid, and any combination thereof.

17. (canceled)

18. The fusion reporter protein of claim 1, further comprising a secretion domain functional in a cell type of interest.

19. The fusion reporter protein of claim 18, wherein the secretion domain is N-terminal to the blocking domain.

20. The fusion reporter protein of claim 18, wherein the cell type of interest is a prokaryotic cell.

21. (canceled)

22. The fusion reporter protein of claim 20, wherein the secretion domain is OsmY or YebF.

23. (canceled)

24. A nucleic acid comprising a sequence encoding the fusion reporter protein recited in claim 1.

25. The nucleic acid of claim 24, further comprising a promoter or enhancer element operatively linked to the sequence encoding the fusion reporter protein.

26. A vector comprising the nucleic acid of claim 24.

27. A system comprising:
a nanopore disposed in a barrier defining a cis side and a trans side, wherein the cis side comprises a first conductive liquid medium and the trans side comprises a second conductive liquid medium, and wherein the nanopore comprises a tunnel that provides liquid communication between the cis side and the trans side;
a data acquisition device operable to detect an ion current through the nanopore; and
a fusion reporter protein of claim 1 in the first liquid medium, wherein a diameter of the blocking domain of the reporter protein exceeds a diameter of the nanopore tunnel at its narrowest point.

28.-32. (canceled)

33. A method of characterizing biological activity of one or more cells in a nanopore system that comprises a nanopore disposed in a barrier defining a cis side and a trans side, wherein the cis side comprises a first conductive liquid medium and the trans side comprises a second conductive liquid medium, and wherein the nanopore comprises a tunnel that provides liquid communication between the cis side and the trans side, the method comprising:
providing a fusion reporter protein as recited in claim 1 into the first conductive liquid medium of the cis side of the nanopore system;
initiating translocation of the flexible tail domain of the fusion reporter protein through the nanopore tunnel, wherein the blocking domain of the fusion reporter protein has a diameter that exceeds the diameter of the nanopore tunnel at its narrowest point;
measuring an ion current between the first conductive liquid medium and the second conductive liquid medium when the flexible analyte domain of the fusion reporter protein is in the tunnel of the nanopore; and
detecting an ion current pattern associated with a structural characteristic of the flexible analyte domain of the fusion reporter protein.

34.-45. (canceled)
